(19) United States
(12) Patent Application Publication
(10) Pub. No.: US 2026/0004920 A1
(43) Pub. Date: Jan. 1, 2026

(54) INTEGRATED HEALTHCARE SUPPLY CHAIN MANAGEMENT METHOD AND SYSTEM UTILIZING MACHINE LEARNING AND ARTIFICIAL INTELLIGENCE FOR ENHANCED DATA ANALYSIS AND DECISION SUPPORT

(71) Applicant: CONCORDANCE INNOVATIONS, LLC, Tiffin, OH (US)

(72) Inventors: Lisa Hohman, Tiffin, OH (US); Cody Fisher, Tiffin, OH (US); Addison Narter-Slezak, Chicago, IL (US); Johan Lars Olof Gauffin, Montreal (CA); David Warren Tessier, JR., West Henrietta, NY (US)

(21) Appl. No.: 19/254,999

(22) Filed: Jun. 30, 2025

Related U.S. Application Data
(60) Provisional application No. 63/665,373, filed on Jun. 28, 2024.

(51) Int. Cl. G16H 40/20; G06Q 10/087

(52) U.S. Cl. CPC G16H 40/20; G06Q 10/087

(57) ABSTRACT

A system for managing healthcare supply chain product and organization information for an organization is provided. The system may include a processor and a memory in communication with the processor. The memory includes a user interface module to receive a product identifier (ID), and an integration module to generate a new product item when the integration module determines that the product ID is not associated with an existing product item, or integrate the product ID with the existing product item when the existing product item does not include the product ID. The memory includes an organization module that receives an organization identifier (ID) and matches the organization ID and organization account to an organization master. The memory includes a PIM module that receives the product item associated with the product ID from the integration module. The PIM module matches the product ID and product item to a system PIM.

### CROSS-REFERENCE TO RELATED APPLICATIONS

This application claims the benefit of U.S. Provisional Application No. 63/665,373, filed on Jun. 28, 2024. The entire disclosure of the above application is incorporated herein by reference.

### FIELD

The present technology relates to product information management for multi-enterprise healthcare supply chains and, more particularly, to standardized data frameworks that enable translation and harmonization of disparate product attributes across organizations while maintaining unique product terminology and identifiers.

### INTRODUCTION

This section provides background information related to the present disclosure which is not necessarily prior art.

Healthcare providers, suppliers, and distributors may face challenges to harmonize and translate inconsistent product and organizational data attributes across different technology systems. Management of healthcare supply chains may involve complex coordination among these entities, leading to inefficiencies such as overstocking, understocking, and delayed responses to important supply needs. These inefficiencies may result in increased costs and compromised patient care, particularly in times of unexpected demand spikes or supply shortages. Healthcare providers, suppliers, and distributors may face difficulties with transactional errors, miscommunication, and administrative inefficiencies, as different systems within the supply chain may possess disparate standards for representing product attributes and organizational details. These systems may impede translation across organizations and impact pricing and contracting processes.

Certain challenges to healthcare supply chain management may include delayed data integration across the different organizations involved. Data may be siloed within each organization, making it difficult to streamline digital product information across enterprises for a comprehensive view of the supply chain status. Absence of a standardized framework for managing and sharing product information may prevent timely decision-making and optimal resource allocation, resulting in discrepancies that further complicate efficient supply chain operations. For example, certain organizations may rely on disparate internal systems for inventory management, procurement, and other supply chain functions.

These systems may be referred to as product information management (PIM) systems, also referred to as an item master. These systems, and the product data within, may either reside in an enterprise resource planning (ERP) software or in additional PIM architecture that acts as the source of truth for all product attributes and information. Organizations using internal language and interpretations, paired with the disparate ERP software or PIM architecture that organizations use to manage the data, may lead to organizations referencing and transacting on inconsistent product attributes and information throughout the healthcare supply chain.

Healthcare organizations may primarily manage product data through a combination of manual processes and ad-hoc solutions, resorting to basic tools such as Microsoft Excel® spreadsheets, to track, manage, and reconcile product data between trading partners. Exchanging data between trading partners may include static files, which is inefficient and prone to error. Organizations may utilize search and organizational methodologies within spreadsheets or pivot tables such as VLOOKUP or XLOOKUP. However, because spreadsheets and pivot tables may not be systematically driven to generate product attribute updates, the data may become unmanageable as the complexity and volume of the data grows within the organization, such as adding new suppliers or managing a larger catalog of products. Suppliers, providers, and distributors may use different naming conventions or data structures, making these search methodologies ineffective. In other words, as organizations grow, maintaining the same manual processes may become increasingly unsustainable. For example, a healthcare provider may be required to manually merge item master information received from a supplier into the internal system of the healthcare provider, which may include the tedious process of mapping product attributes such as item codes, descriptions, and supplier information. Challenges to manual integration may be magnified by fragmented systems within each organization that do not communicate well with each other. For example, product data may be stored in multiple locations and in formats, e.g., Enterprise resource planning (ERP), inventory management, procurement systems, etc. Without a centralized, automated system to manage product data, organizations may experience delayed orders, inventory shortages, contract and pricing challenges, and other supply chain disruptions.

Reliance on manual data entry and analysis may be time-consuming, require substantial human intervention, and prone to errors. The absence of automated systems to facilitate seamless data translation across systems may hinder an organization in identifying duplicate product entries, e.g., a single item may be added multiple times due to slightly different description and attributes, leading to multiple records for the same product in the same system. Without a robust system to detect and resolve these duplicates, healthcare organizations may struggle to maintain a clean, consolidated item master. Similarly, product attributes may be missing from product entries and are necessary for organizations to make informed decisions and communicate across trading partners. Manual transference of data may include fragmented communication between trading partners such as file exchanges through email threads. This communication may not only slow the supply chain but also require skilled personnel to handle complex data reconciliation tasks.

Certain organizations may attempt to militate against these challenges by outsourcing data enrichment and harmonization to third parties. Third-party providers may offer one-way feeds of product information where data flows from the provider to the organization without reciprocal updates or active integration. Third-party data providers, however, may not offer full integration with other organization systems, meaning organizations may still rely on internal manual processes to incorporate the external data into their internal workflows, increasing the potential for errors and delays. These feeds may be static where the product data remains unchanged until a new feed is issued, often at fixed intervals that may not capture real-time adjustments in product catalogs, such as changes in description availability, or packaging. Changes in the supplier's product catalog, e.g., discontinued items, new products, or updated product attributes may go unnoticed for a period of time, leading to further discrepancies. Third-party providers may also face challenges with the wide variety of formatting, quality, and completeness of data acquired from different organizations. For example, different suppliers may use different naming conventions, product classifications, or measurement units, resulting in inconsistencies when data from multiple providers is merged. If the data is not sourced from a trusted organization or a direct channel, there may be discrepancies or gaps in product information that cannot be easily resolved without manual intervention.

When using certain third-party providers, an organization may rely on the third-party provider for the accuracy or quality of the data the organization receives. If the data is incorrect or inconsistent, it may be difficult to resolve issues in a timely manner without significant back-and-forth with the third-party provider. Organizations may not fully own the data that they receive from third-party providers. In other words, the organization may not possess the authority to add or update data in ways that fit the specific business requirements of the organization. Third-party solutions may not be able to adapt quickly enough to new trends, market changes, or technological innovations, leaving organizations reliant on outdated systems or non-compliant data in a rapidly evolving industry. The inability to update inconsistent or outdated product data may result in compliance issues, particularly where regulatory standards and safety protocols are constantly evolving.

There is a continuing need for an enhanced healthcare supply chain management system that enriches, harmonizes and aligns product information across multiple enterprises within the healthcare supply chain. Desirably, such a system would offer robust real-time data integration, advanced matching analytics, and effective collaboration tailored to the specific needs of the healthcare industry. Addressing these issues may allow for greater consistency in product attributes and organizational data, providing efficiency and reliability to healthcare supply chain operations and ensuring the availability of necessary medical supplies to healthcare providers.

### SUMMARY

In concordance with the instant disclosure, an enhanced healthcare supply chain management system that enriches, harmonizes and aligns product information across multiple enterprises within the healthcare supply chain, has been discovered.

The present technology includes systems and processes that relate to the optimization of healthcare supply chain management through advanced data integration, real-time analytics, and the strategic application of machine learning and artificial intelligence to enhance operational efficiency and decision-making across healthcare entities. It enhances decision-making processes through the use of machine learning and artificial intelligence, enabling healthcare providers to anticipate supply needs, mitigate risks of shortages or surpluses, and ensure the timely availability of essential medical supplies. This technology significantly reduces operational costs and improves patient care by optimizing the flow of goods and information across the healthcare supply chain.

In certain embodiments, a system for managing a healthcare supply chain for an organization is provided. The system may include a processor and a memory in communication with the processor. The memory may include a user interface module that receives a product identifier (ID) and an organization identifier (ID) from the organization. The memory may include an integration module that receives the product ID and organization ID from the user interface module. The integration module may generate a new product item when the integration module determines that the product ID is not associated with an existing product item. The integration module may generate a new organization account when the integration module determines that the organization ID is not associated with an existing organization account. The integration module may integrate the product ID with the existing product item when the existing product item does not include the product ID. The integration module may integrate the organization ID with the existing organization account when the existing organization account does not include the organization ID. The memory may include an organization module that receives the organization ID associated with the organization ID from the integration module. The organization module may match the organization ID and organization account to an organization master. The memory may include a PIM module that receives the product item associated with the product ID from the integration module. The PIM module may match the product ID and product item to a system PIM.

In certain embodiments, the memory may include an artificial intelligence (AI) module that receives the product ID from the integration module. The AI module may retrieve two or more existing product items that include a partial match to the product ID. The AI module may integrate the product ID with one of the two or more existing product items. The AI module may include a matching algorithm configured to generate a confidence score based on the partial match between the product ID and the two or more existing product items. The learning engine may match the product ID to one of the two or more existing product items based on the confidence score.

In certain embodiments, a method for managing a healthcare supply chain for an organization is provided. The method may operate in conjunction with a system for managing a healthcare supply chain for an organization, as described herein. The method may include a step of receiving the product ID and the organization ID from the organization. The method may include a step of generating the new product item when the integration module determines that the product ID is not associated with an existing product item. The method may include a step of generating the new organization account when the integration module determines that the organization ID is not associated with an existing organization account. The method may include a step of integrating the product ID with the existing product item when the existing product item does not include the product ID. The method may include a step of integrating the organization ID with the existing organization account when the existing organization account does not include the organization ID. The method may include a step of matching the organization ID and organization account to an organization master. The method may include a step of matching the product ID and product item to a system PIM.

In certain embodiments, a non-transitory computer-readable medium, operable to store processor instructions for managing a healthcare supply chain for an organization is provided. When executed by a processor, the processor instructions may cause the processor to receive a product ID and an organization ID from the organization. The processor instructions may cause the processor to generate a new product item when an integration module determines that the product ID is not associated with an existing product item. The processor instructions may cause the processor to generate a new organization account when the integration module determines that the organization ID is not associated with an existing organization account. The processor instructions may cause the processor to integrate the product ID with the existing product item when the existing product item does not include the product ID. The processor instructions may cause the processor to integrate the organization ID with the existing organization account when the existing organization account does not include the organization ID. The processor instructions may cause the processor to match the organization ID and organization account to an organization master. The processor instructions may cause the processor to match the product ID and product item to a system PIM.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a block diagram illustrating a healthcare supply chain management system, according to some embodiments of the present disclosure;

FIG. 2 is a block diagram illustrating a healthcare supply chain management system, according to some embodiments of the present disclosure;

FIG. 3 is a block diagram illustrating a healthcare supply chain management system, according to some embodiments of the present disclosure;

FIG. 4 illustrates a graphical user interface for cross-referencing product items in a healthcare supply chain management system;

FIG. 5 illustrates a graphical user interface for viewing product information management (PIM) data through an item master in a healthcare supply chain management system;

FIG. 6 illustrates a graphical user interface for viewing PIM data through an item master in a healthcare supply chain management system;

FIG. 7 illustrates a graphical user interface for viewing substitute product items in a healthcare supply chain management system;

FIG. 8 is a block diagram illustrating the relationship between the item master and organizations in healthcare supply chain management system, according to some embodiments of the present disclosure;

FIGS. 9A-9D are a sequence diagram illustrating a healthcare supply chain management system, according to some embodiments of the present disclosure;

FIGS. 10A and 10B are entity relationship diagrams illustrating the relationship between the item master and the organization master in a healthcare supply chain management system, according to some embodiments of the present disclosure;

FIGS. 11A and 11B provide a flowchart illustrating an embodiment of a method for managing a healthcare supply chain product and organization information for an organization;

FIG. 12 provides a flowchart extending from FIGS. 11A and 11B and further illustrates a method for managing healthcare supply chain product and organization information for an organization;

FIG. 13 provides a flowchart extending from FIGS. 11A and 11B and further illustrates a method for managing a healthcare supply chain product and organization information for an organization;

FIG. 14 provides a flowchart extending from FIGS. 11A and 11B and further illustrates a method for managing a healthcare supply chain product and organization information for an organization;

FIG. 15 provides a flowchart extending from FIGS. 11A and 11B and further illustrates a method for managing a healthcare supply chain product and organization information for an organization;

FIG. 16 provides a flowchart extending from FIGS. 11A and 11B and further illustrates a method for managing a healthcare supply chain product and organization information for an organization; and

FIGS. 17A and 17B provide a flowchart illustrating an embodiment of a method for managing a healthcare supply chain product and organization information for an organization.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

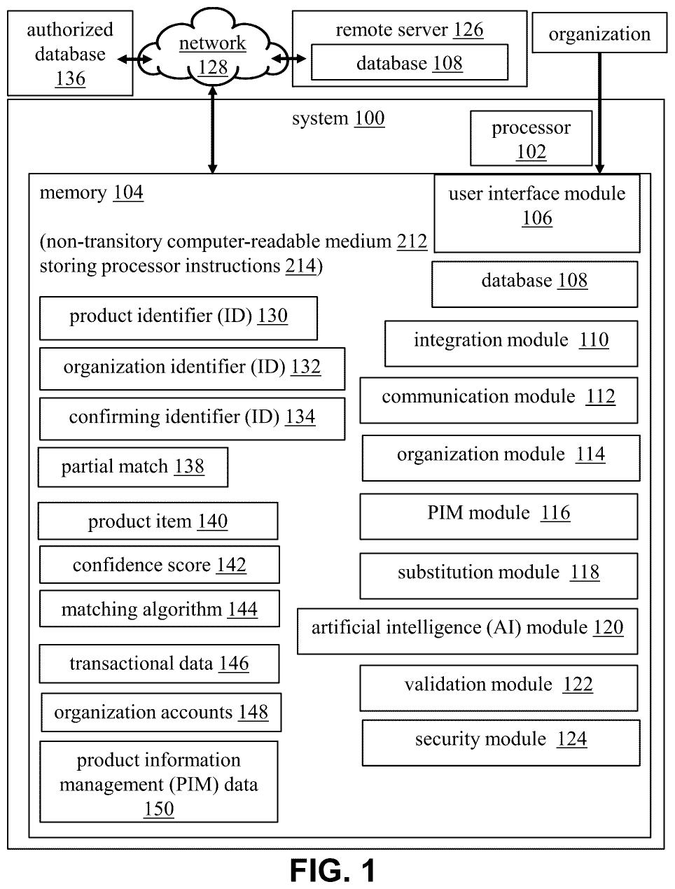

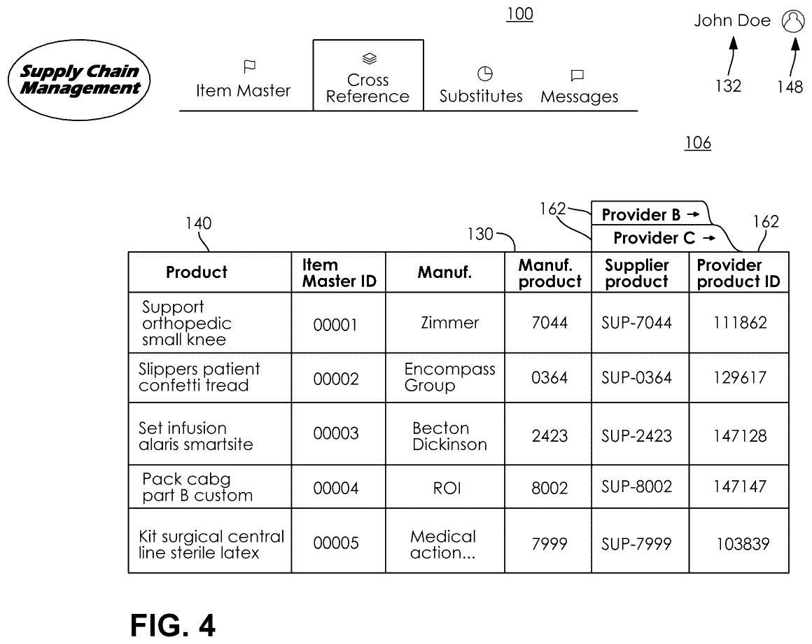

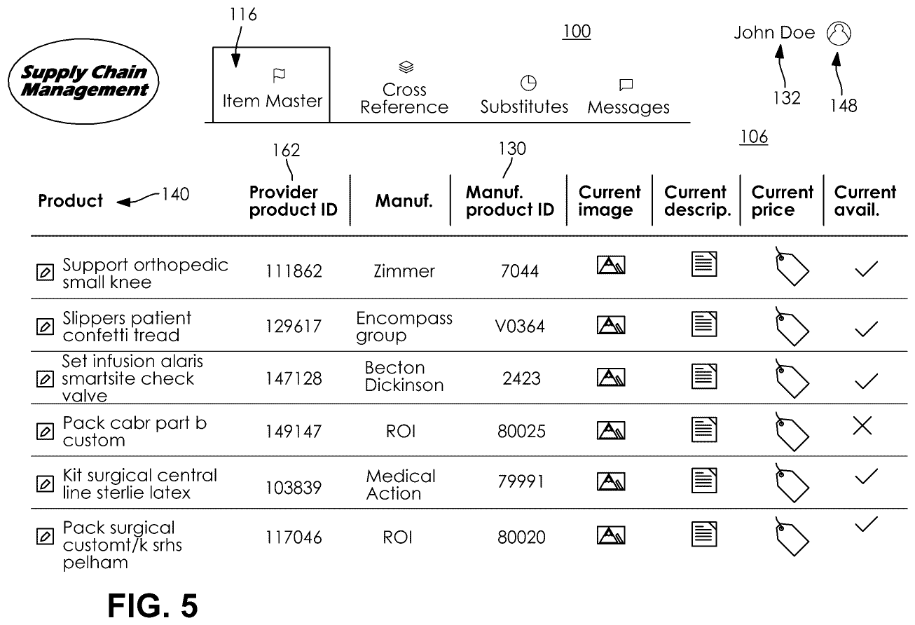

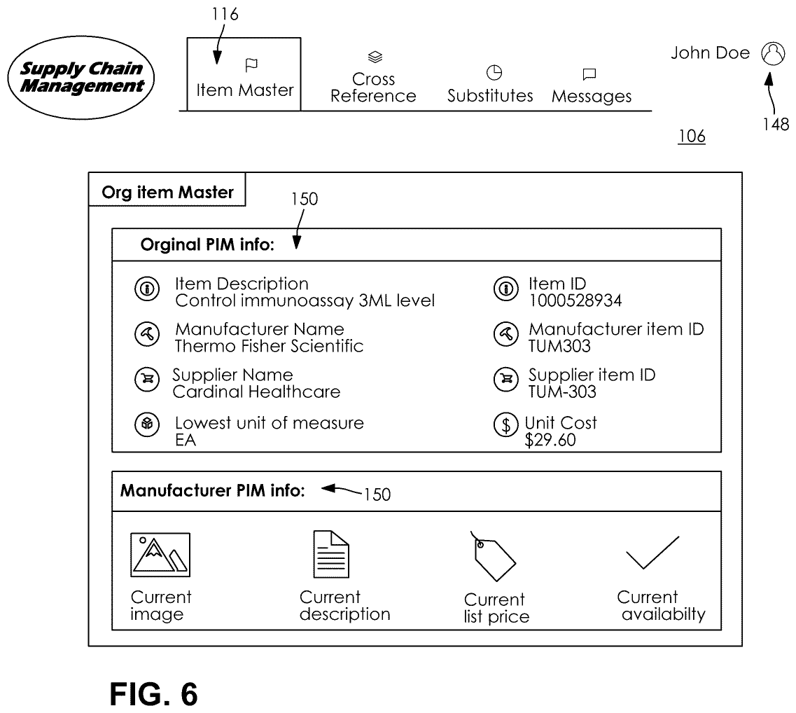

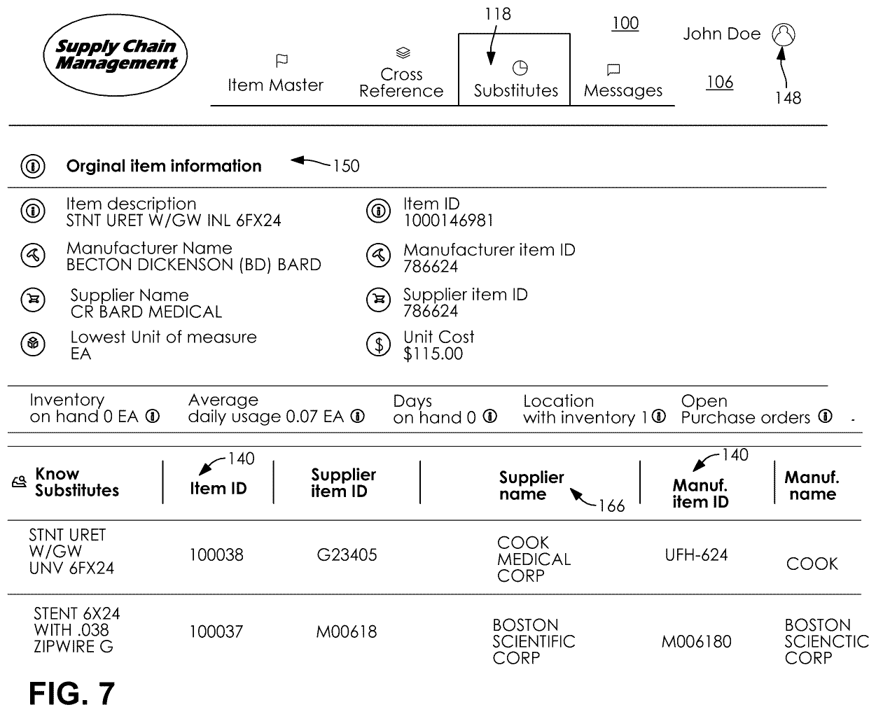

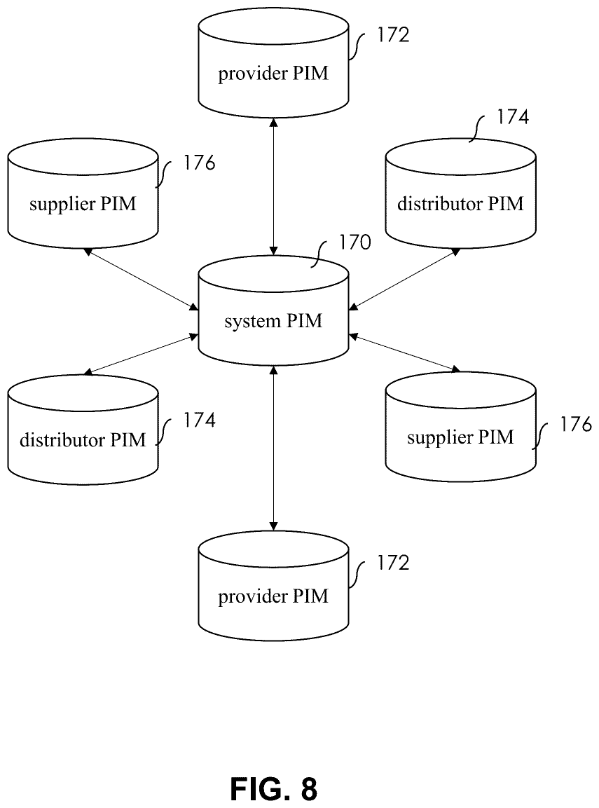

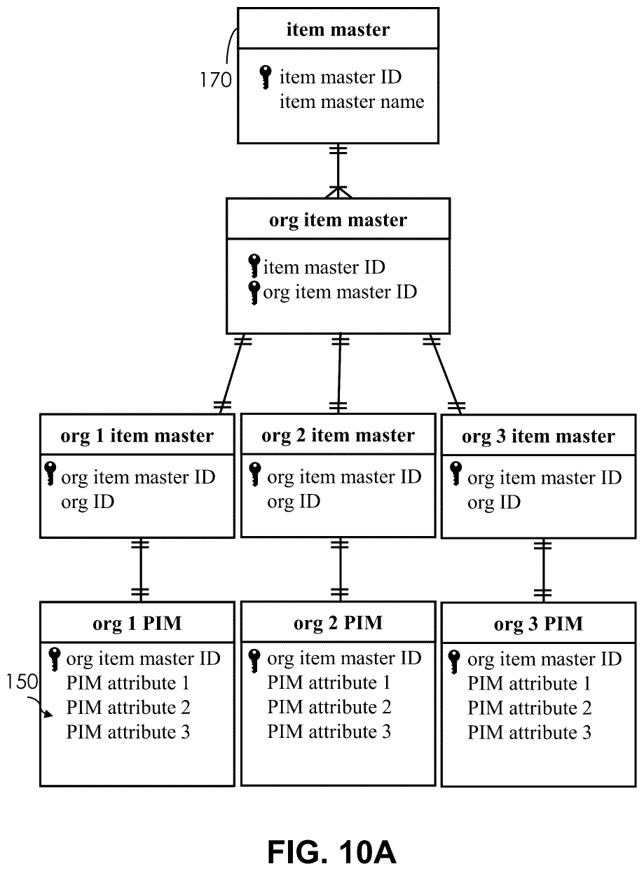

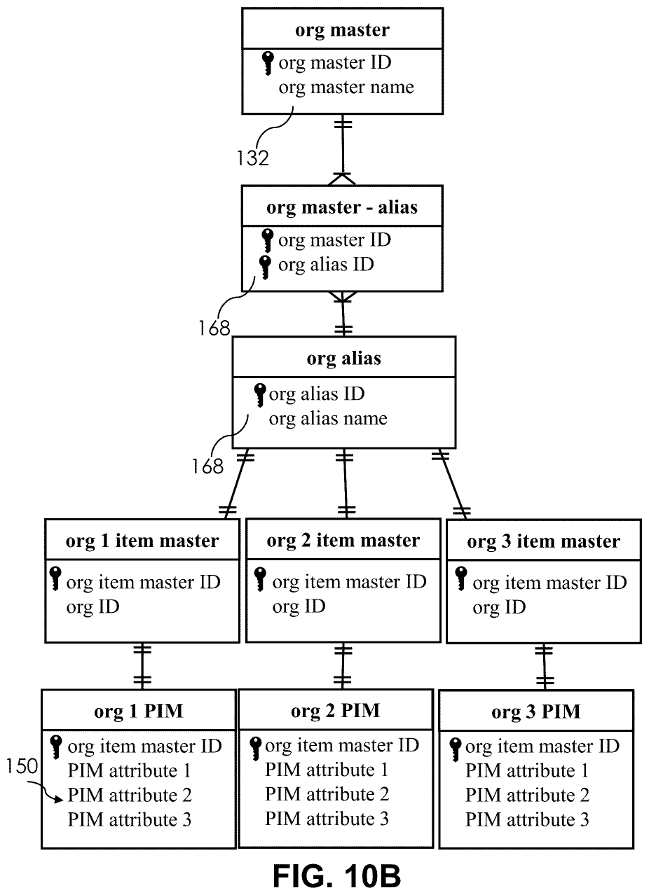

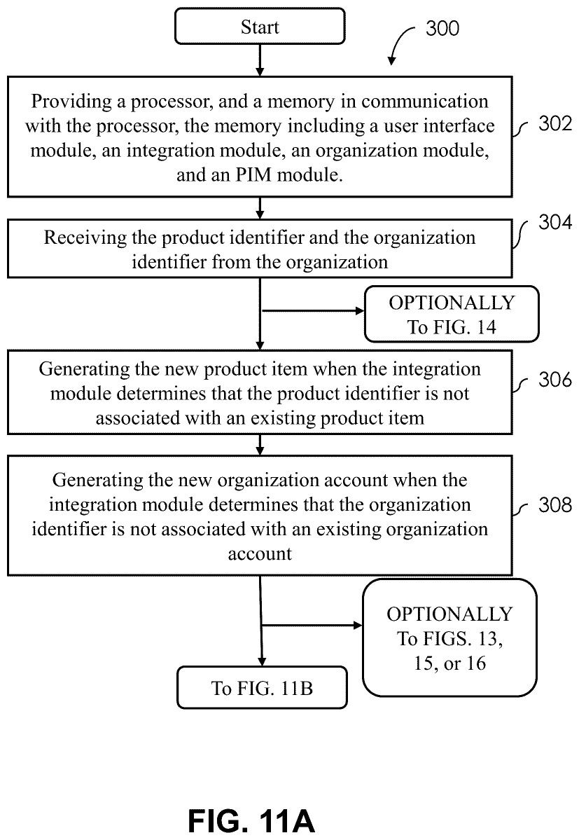

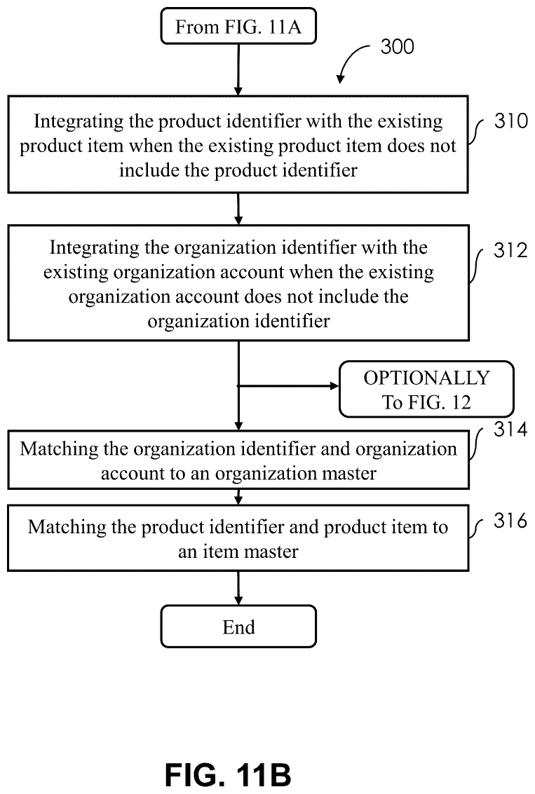

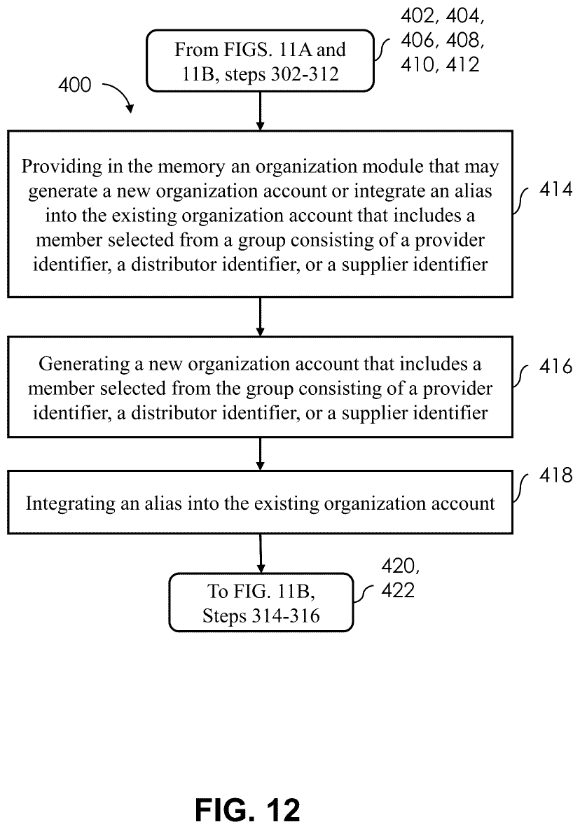

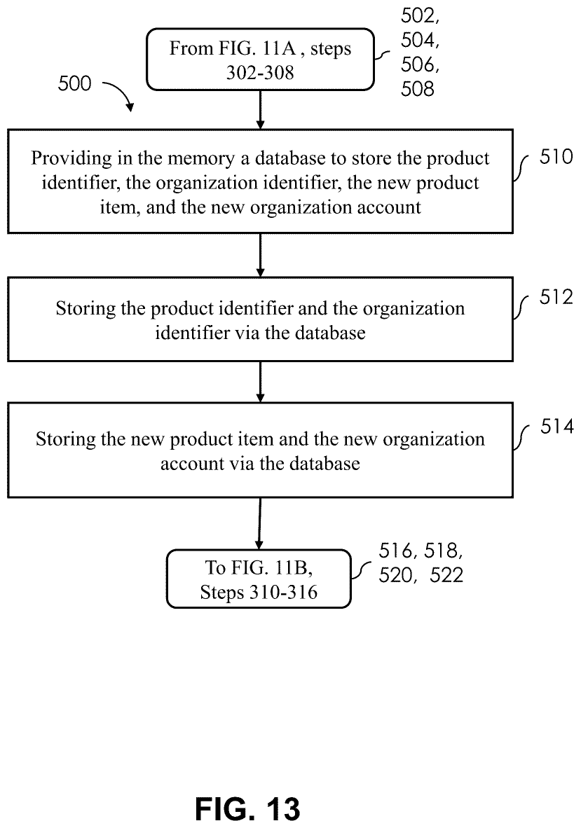

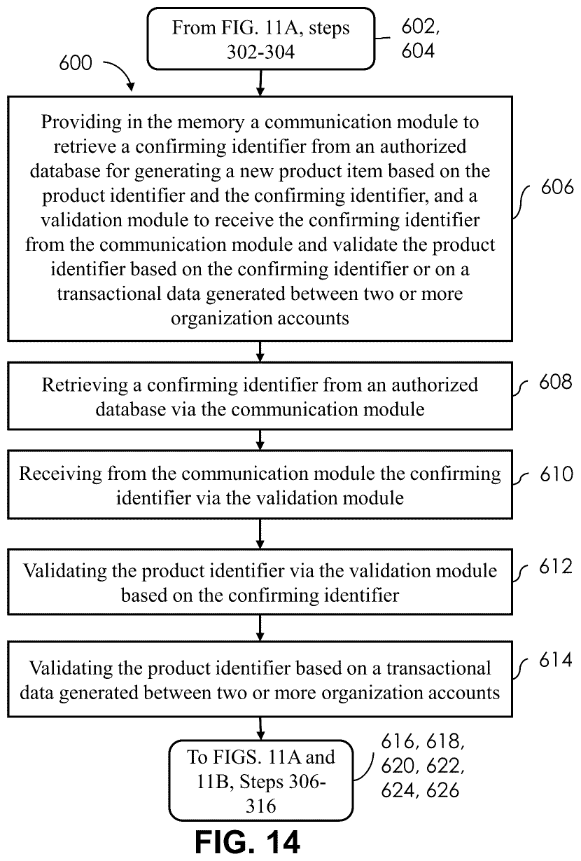

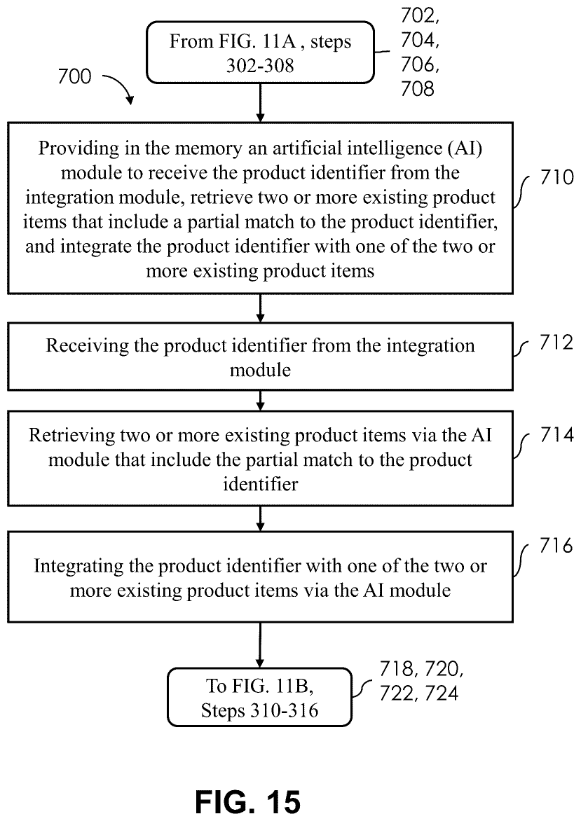

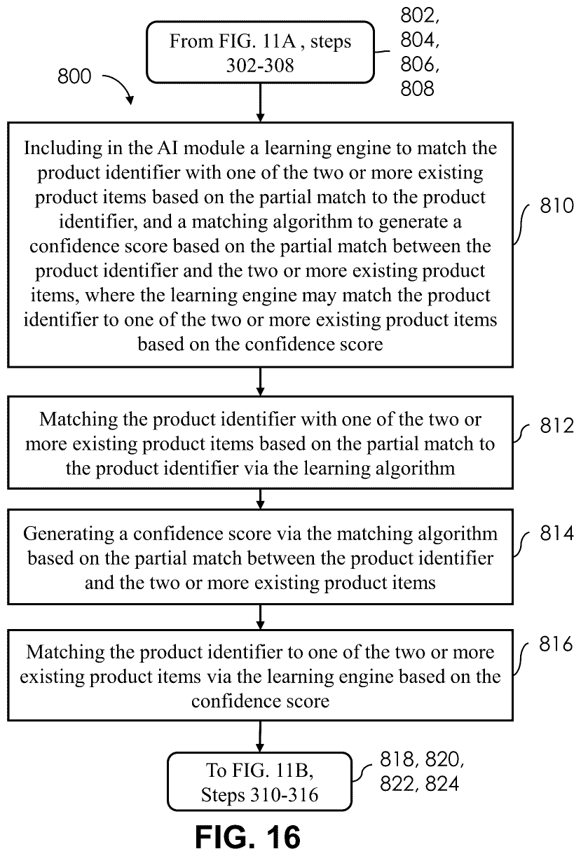

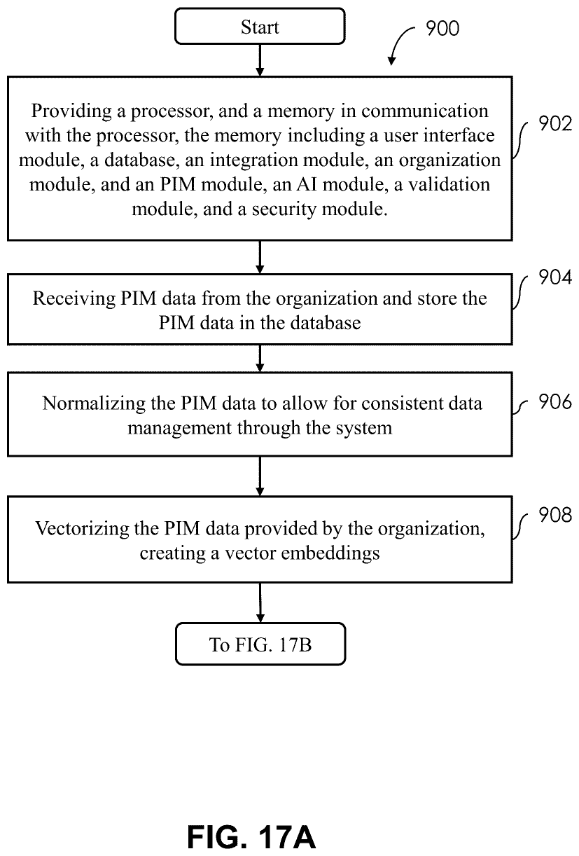

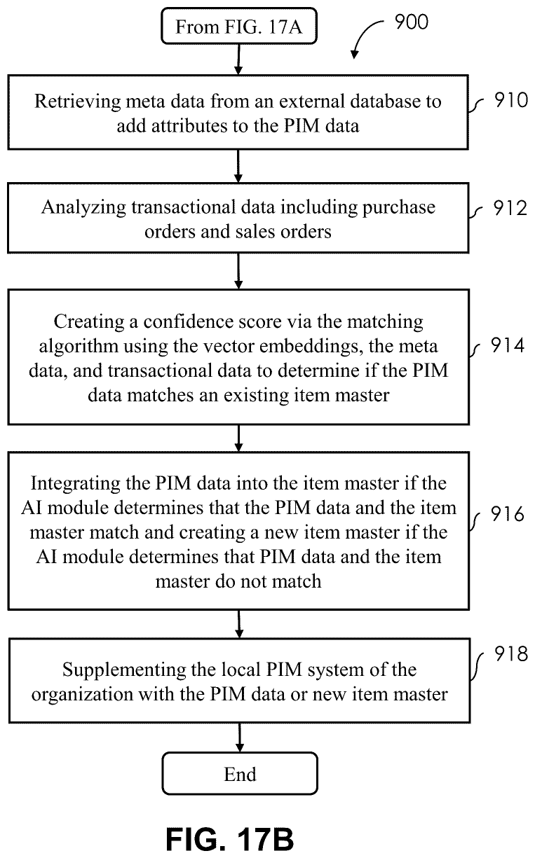

The present technology provides an advanced system 100 for improved responsiveness and efficiency of healthcare supply chain management product and organization information by utilizing real-time data integration with artificial intelligence and machine learning to match product information across disparate healthcare management systems, aspects of which are shown generally in accompanying FIGS. 1-10B. A method 300 for managing healthcare supply chain product and organization information for an organization is also disclosed, aspects of which are shown in FIGS. 11A and 11B. Another method 400 for managing healthcare supply chain product and organization information for an organization is disclosed in FIG. 12. Another method 500 for managing healthcare supply chain product and organization information for an organization is disclosed in FIG. 13. And another method 600 for managing healthcare supply chain product and organization information for an organization is also disclosed in FIG. 14. Another method 700 for managing healthcare supply chain product and organization information for an organization in FIG. 15. Yet another method 800 for managing healthcare supply chain product and organization information for an organization is disclosed in FIG. 16. And yet another method 900 for managing a healthcare supply chain for an organization is disclosed in FIGS. 17A and 17B.

The system 100 and methods 300, 400, 500, 600, 700, 800, and 900 allow an organization to enrich, harmonize and align local product data with product data from other organizations within a healthcare supply chain. As shown in FIGS. 1-10B, the system 100 may include a processor 102 and a memory 104 in communication with the processor 102. The memory 104 may include a user interface module 106, a database 108, an integration module 110, a communication module 112, an organization module 114, a product information management (PIM) module 116, a substitution module 118, an artificial intelligence (AI) module 120, a validation module 122, and a security module 124.

The processor 102 may be located on a local system 100 or a remote server 126 accessed via a network 128. The remote server 126 may be the central hub of the system 100, containing the processor 102 and memory 104 that store and execute the modules necessary for processing data. One skilled in the art will also appreciate that the processor 102 may include one or more processors and may process information and execute the various instructions or operations, as described herein. For example, the processor 102 may include a central processing unit (CPU), a microprocessor, a microcontroller, a system-on-a-chip 100, a digital signal processor (DSPs), field-programmable gate arrays (FPGAs), application-specific integrated circuits (ASICs), and/or a processor based on a multi-core processor architecture. One or more processors 102 may mean a single processor or multiple processors in a single processing unit, e.g., a central processing unit, or multiple processing units, e.g., a central processing unit and a graphics processing unit, or a central processing unit and a memory 104 manager. The processor 102 may include multiple processors 102 where one processor 102 is capable of executing one or more of the elements described in this disclosure, and a subsequent processor 102 or processors 102 may execute other elements as described herein, capable of executing all elements only in combination. One or more of the processors 102 may be remote from the at least one local system 100 server.

The memory 104 may store or otherwise include one or more databases 108. The memory 104 can include one or more memories and of any type suitable to the system 100 and can be implemented using any suitable volatile or nonvolatile data storage technology such as a semiconductor-based memory device, a magnetic memory device, an optical memory, a fixed memory, and/or a removable memory. For example, the memory 104 may include any combination of random-access memory (RAM), read only memory (ROM), static storage such as a magnetic or optical disk, a hard disk drive (HDD), or any other type of non-transitory machine or computer readable media.

As shown in FIGS. 1 and 4-7, the user interface module 106 may serve as an interface for the system 100. The user interface module 106 may serve as the point of interaction between a user and the system 100 and interact with hardware including various output devices that may display a representation of the user interface module 106 for observation by the user, where such an output device may include, for example, one or more computer screen, speaker, tablet screen, or other view/audio port. The user interface module 106 may include, for example, a graphical user interface (e.g., an XR interface or XR space) that can be displayed in various ways, for example, via a desktop application, smartphone or mobile application, web interface, or API, and may interface with mobile SMS, social platforms, or messaging applications. The user interface module 106 may be designed to be intuitive and user-friendly, for example, with custom user preferences and accessibility requirements, allowing the organization to easily upload, type, or choose a retrieved or generated product identifier (ID) 130 or an organization identifier (ID) 132. Alternatively, the user interface module 106 may receive a product item 140 from the organization if the organization does not possess the product ID 130. The user interface module 106 may receive the product ID 130 or the organization ID 132 from the user for further processing by system 100, and for use in the integration module 110.

As shown in FIG. 1, the database 108 may receive and store the product ID 130 or the organization ID 132, or store data relating to a new product item 140 or a new organization account 148. The database 108 may store a confirming identifier (ID) 134 from an authorized database 136. The database 108 may also store information retrieved or generated from the integration module 110, the organization module 114, the PIM module 116, the AI module 120, or the validation module 122. For example, the database 108 may store data relating to a partial match 138 between a product ID 130 and existing product items 140 or a confidence score 142 generated by the matching algorithm 144. The database 108 may also store transactional data 146 generated between organization accounts 148.

The database 108 may include a local database 108 as shown in FIG. 1, option 1, a database 108 saved on a remote server 126 and accessed via a network 128, as shown in FIG. 1, option 2, such as a cloud server, or a combination of a local and a remote database 108, as required by the system 100. The database 108 may also include, for example, a vector database 108 or vector store for storing vector embeddings 202, e.g. flexible, meaning-based, probabilistic numerical representations of data that capture semantic meaning, allowing the system 100 to compare similarities between different types of data. The database 108 may also include a relational database 108, for example, data saved in a structured form, e.g. a structured query language (SQL) table, a comma-separated values (CSV) file, or in JavaScript object notation (JSON), or a JSON-related object or map, or object storage, or other forms of tabular input. The database 108 may include normalized product information management (PIM) data 150. The database 108 may also include a general storage database 108 to store, for example, unstructured data such as HTML, text, raw transcripts, chat logs, images, audio files, or social media posts. It should be understood that the database 108 may employ a separate or secondary encryption to protect sensitive information, ensuring that the stored data remains secure and confidential.

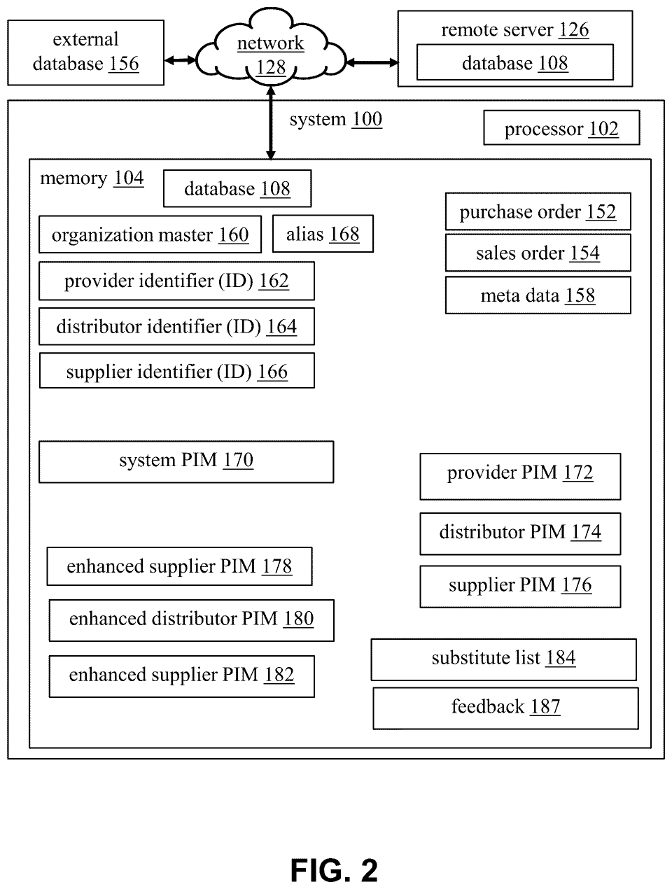

With reference to FIGS. 1-2, the integration module 110 may receive the product ID 130 and organization ID 132 from the user interface module 106. The integration module 110 may generate a new product item 140 when the integration module 110 determines that the product ID 130 is not associated with an existing product item 140. The integration module 110 may generate a new organization account 148 when the integration module 110 determines that the organization ID 132 is not associated with an existing organization account 148. The integration module 110 may integrate the product ID 130 with the existing product item 140 when the existing product items 140 does not include the product ID 130. The integration module 110 may integrate the organization ID 132 with the existing organization account 148 when the existing organization account 148 does not include the organization ID 132. The integration module 110 may allow for the integration of a purchase order 152, a sales order 154, inventory and other supply chain information. It should be appreciated that the integration of both purchase orders 152 and sales orders 154 may enable organizations to achieve a higher level of confirmation and validation of information on a product item 140 from an entire transaction lifecycle.

As shown in FIGS. 1 and 2, the communication module 112 may retrieve a confirming ID 134 from an authorized database 136 for generating a new product item 140 based on the product ID 130 and the confirming ID 134. The communication module 112 may communicate with an external database 156, for example, the Food and Drug Administration (FDA), Data Universal Numbering System (DUNS), and utilize universal serial numbers such as a Global Location Number (GLN), Unique Entity ID (UEI). For example, the FDA may provide information relating to drugs, medical devices, and other healthcare-related products, and may maintain several external databases 156 that contain detailed, accurate information about regulated products. These external databases 156 may enrich product information in the system 100 and in other PIM systems, such as the FDA 510(k) Database, the FDA Drug Approval Database, and the FDA National Drug Code (NDC) Directory. Other external databases 156 from third-party sources may be utilized, such as the GS1 Global Data Synchronization Network (GDSN). The communication module 112 may retrieve, for example, universal serial numbers such as a GS1 Global Trade Item Number (GTIN), a Global Location Number (GLN), or other unique identifiers to help track product items 140 across the supply chain and obtain detailed PIM data 150 such as descriptions, dimensions, packaging information, barcode data, and regulatory compliance across regions.

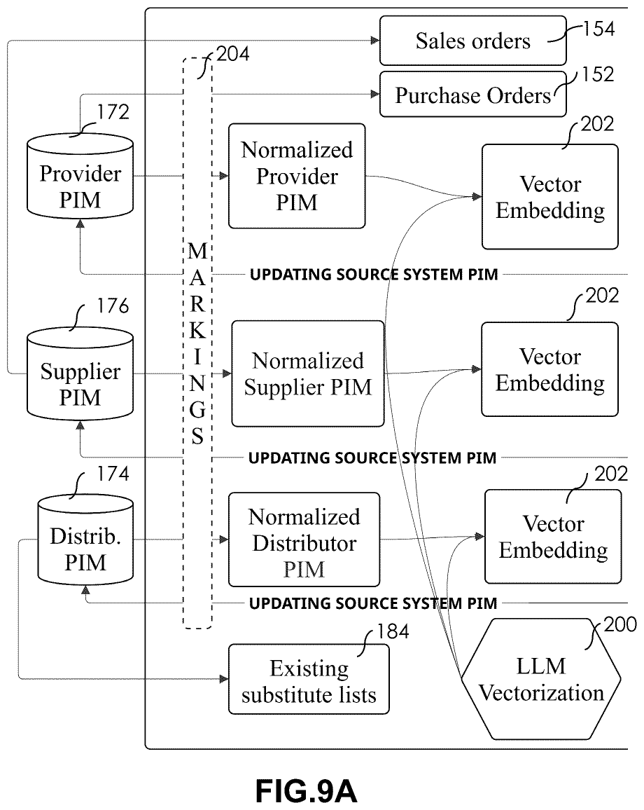

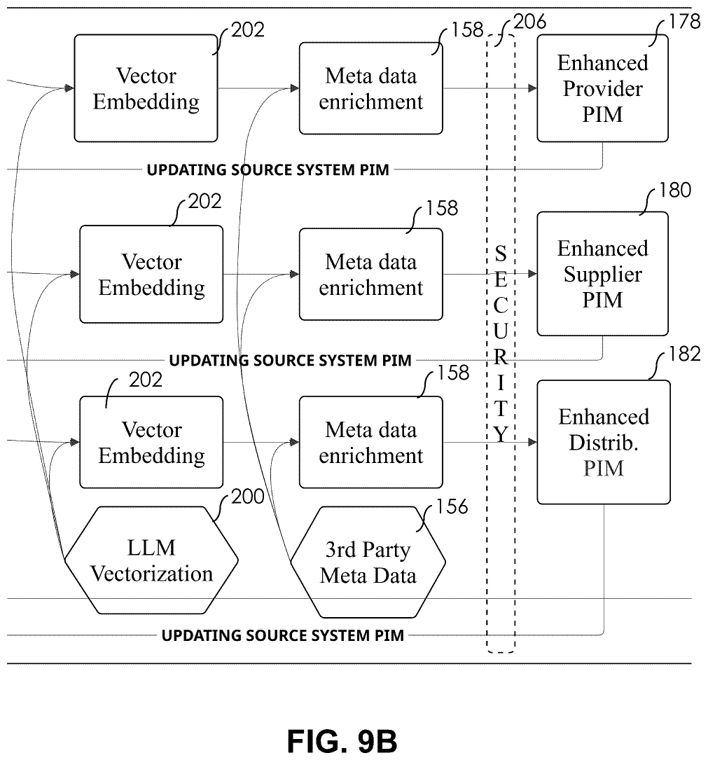

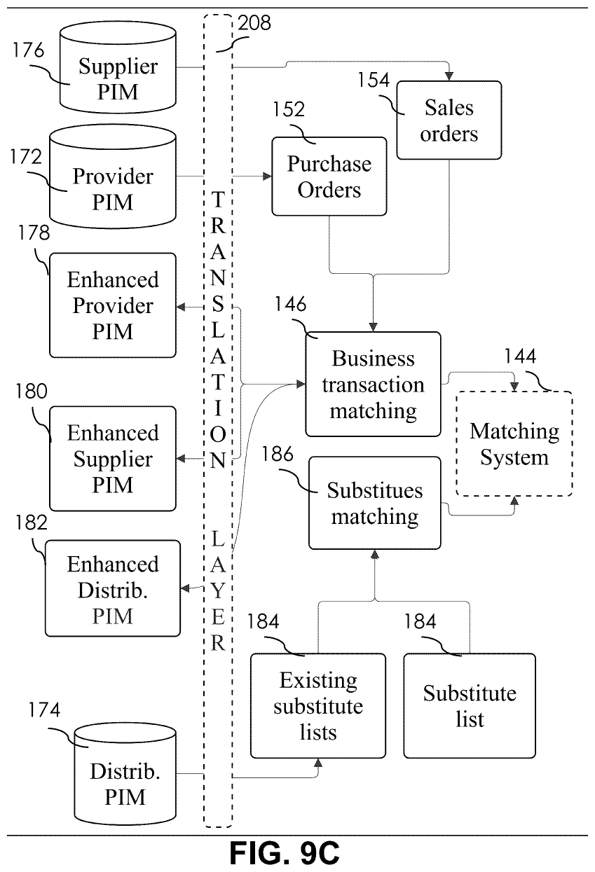

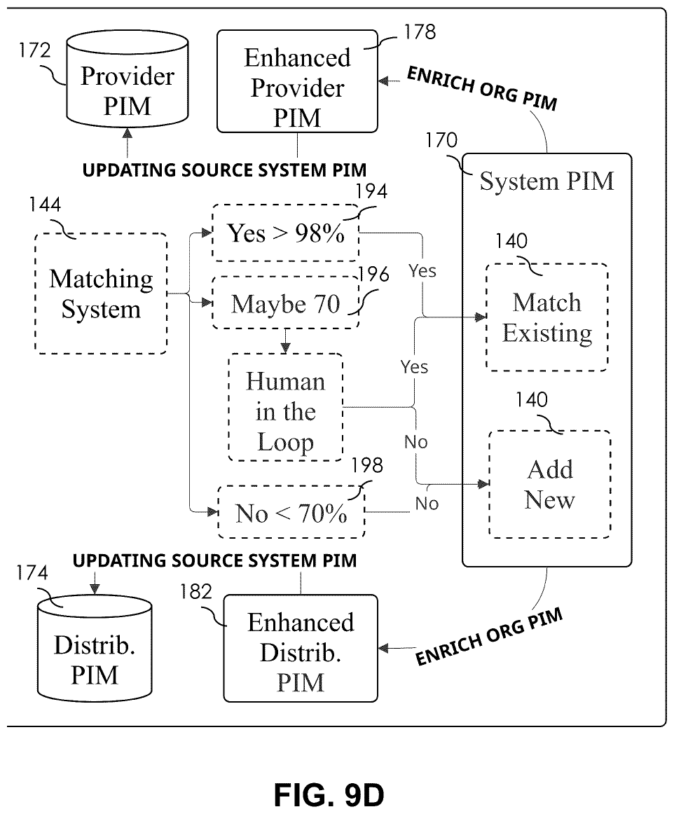

As shown in FIGS. 1 and 9B, the communication module 112 may allow information from the external database 156 to enrich normalized PIM data 150 by providing additional information, including third-party meta data 158, embedding the meta data 158 directly into the PIM data 150. It should be appreciated that the meta data 158 may allow for each product item 140 in the system 100 to be enriched with essential external data that enhances its quality and compliance. To ensure that the enriched meta data 158 remains current, the communication module 112 may automate communication with an external database 156. As the meta data 158 changes, e.g., a new certification is issued or the compliance status of a product item 140 is updated, the PIM data 150 may be enhanced on a regular schedule depending on the attributes of the PIM data 150 and the criticality of the PIM data 150 to the system 100. It should be appreciated that the external database 156 may provide supplementary information that can be integrated into the PIM system of an organization to enrich existing PIM data 150, enhancing the quality, compliance, and traceability of PIM data 150 by adding authoritative and up-to-date details from trusted external repositories.

As shown in FIGS. 1 and 2, the organization module 114 may receive the organization ID 132 from the integration module 110. The organization module 114 may match the organization ID 132 and organization account 148 to an organization master 160. The organization module 114 may generate a new organization account 148 that may include a provider identifier (ID) 162, a distributor identifier (ID) 164, or a supplier identifier (ID) 166. The provider ID 162 may include supplier IDs 166 and aliases 168 associated with supplier IDs 166 associated with the provider ID 162. The distributor ID 164 may include supplier IDs 166, provider IDs 162, and aliases 168 associated with supplier IDs 166 and provider IDs 162. The supplier ID 166 may include distributor IDs 164, providers ID 162, and aliases 168 associated with distributor IDs 164 and provider IDs 162.

The organization module 114 may also integrate an alias 168 into the existing organization account 148, for example, an alias 168 for a provider ID 162, a distributor ID 164, or a supplier ID 166. For example, the organization module 114 may map variations of an organization ID 132 to one unique organization ID 132 or existing organization account 148, e.g., the organization “Minnesota Materials Manufacturing” may have several aliases 168 including “3M”, “MMM”, and “Three M”.

As shown in FIGS. 1 and 9C-9D, the organization module 114 may also allow for the system 100 to provide an enhanced provider product information management (PIM) 178, enhanced supplier product information management (PIM) 180, and enhanced distributor product information management (PIM) 182, which may be consistently enriched and updated in real time with PIM data 150, enabling cross-organization collaboration and maintaining maximum data integrity at the organizational level. The enhanced provider PIM 178, enhanced supplier PIM 180 and enhanced distributor PIM 182 may act as intermediaries between the PIM module 116 and the PIM system of the organization residing outside of the system 100. It should be appreciated that the integration of the enhanced provider PIM 178, enhanced supplier PIM 180, and enhanced distributor PIM 182 with disparate systems may allow organizations to consistently update, enrich, and synchronize internal PIM data 150.

As shown in FIGS. 2 and 5-6, the PIM module 116 may receive the product item 140 associated with the product ID 130 from the integration module 110. The PIM module 116 may match the product ID 130 and product item 140 to a system product information management (PIM) 170. The PIM module 116 may serve as a centralized repository that manages all PIM data 150, attributes, and meta data 158 for a product item 140 within the system 100. The PIM module 116 may retain comprehensive product information, ensuring consistency, accuracy, and seamless integration across multiple organizations within the system 100. The system 100 may serve as the single source of truth for product information, enabling organizations to access and share product information efficiently. In other words, each product item 140 is provided a product ID 130, e.g., a unique key, creating a consistent identifier that links the product information across external PIM systems. It should be appreciated that the unique product ID 130 may allow for the accurate translation of PIM data 150 from one organization to another, eliminating inconsistencies and streamlining cross-organization data flow.

The PIM module 116 may include a system product information management (PIM) 174, and a supplier product information management (PIM) 170, as well as a provider product information management (PIM) 172, a distributor product information management (PIM) 174, and a supplier product information management (PIM) 176. The provider PIM 172 may provide a centralized database 108 for the system 100 to manage PIM data 150 for a product item 140. The provider PIM 172 may serve as a single source of truth for all attributes and other data points of the product item 140 to allow consistency and accuracy, for example, when ordering, managing, and using the product item 140. For example, the provider PIM 172 may include PIM data 150 such as a product ID 130, among other attributes as shown in Table 1A. The provider PIM 172 may include one or more of the following attributes listed in Table 1A:

TABLE 1A
Description of PIM data 150 attributes for use with the provider PIM 172.

| No. | Attribute Name | Description |
|---|---|---|
| 1 | Provider Item ID | Unique identifier assigned to each product in the healthcare |
| | | system (e.g., SKU, internal catalog number) |
| 2 | Provider Product | The official name of the product, typically standardized across |
| | Name | organizations (e.g., “Surgical Mask, Type IIR”) |
| 3 | Provider Product | A detailed description of the product, including its intended |
| | Description | use, features, and other relevant details |
| 4 | Manufacturer | The name of the manufacturer that produces the product |
| | Name | |
| 5 | Manufacturer Part | A unique identifier assigned by the manufacturer to the |
| | Number (MPN) | product |
| 6 | Supplier Name | The name of the supplier responsible for distributing the |
| | | product |
| 7 | Supplier Part | The unique identifier used by the supplier to catalog the |
| | Number (SPN) | product |
| 8 | Supplier Contact | Details like phone number, email, and address of the supplier |
| | Information | for procurement and support |
| 9 | Brand Name | The brand associated with the product, especially when there |
| | | are multiple brands for the same type of product |
| 10 | Product Category | The general classification of the product (e.g., “Medical |
| | | Devices”, “Surgical Supplies”) |
| 11 | Product | A more specific categorization within the broader category |
| | Subcategory | (e.g., “Personal Protective Equipment”, “Face Masks”) |
| 12 | Product | A broader grouping of products, often used for aggregation in |
| | Type/Family | reports (e.g., “Surgical Instruments”, “Orthopedic Devices”) |
| 13 | Product | Physical measurements (e.g., length, width, height, weight) of |
| | Dimensions | the product |
| 14 | Unit of Measures | The unit of measure used to quantify the product (e.g., “Each”, |
| | (UOM) | “Box”, “Carton”, “Pallet”) |
| 15 | Global Trade Item | A unique identifier used to track trade items. GTINs (e.g., |
| | Number (GTIN) | GTIN-13, GTIN-14) are critical for product identification |
| | | across systems (e.g., barcode scanning) |
| 16 | United Nations | A unique numerical identifier that represents a specific |
| | Standard Products | category of products or services in the UNSPSC classification |
| | and Services | system utilizing a four-level hierarchical structure, with each |
| | (UNSPC) Code | level corresponding to a more specific categorization of goods |
| | | or services |
| 17 | UNSPC | Used alongside the UNSPC Code to describe the goods or |
| | Description | services in layman's terms or a broader industry context, |
| | | identifying products within procurement systems, inventory |
| | | systems, and supply chain management tools, as the code |
| | | alone might not fully describe the product |
| 18 | Country of Origin | The country where the product was manufactured or sourced |
| | | from (e.g., “China”, “USA”) |
| 19 | Expiration Date | For medical products with shelf lives, the expiration date is |
| | | critical to tracking product usability and compliance |
| 20 | Shelf Life | The duration for which the product is considered safe and |
| | | effective |
| 21 | Product Status | Whether the product is “Active”, “Discontinued”, or |
| | | “Obsolete”. The product status helps in tracking products that |
| | | are no longer available or supported |
| 22 | Regulatory | Indicates whether the product complies with relevant |
| | Compliance Status | regulations (e.g., FDA approval for medical devices) |
| 23 | FDA Classification | For healthcare products, this classification specifies the FDA |
| | | regulatory category (e.g., Class I, Class II, Class III) |
| 24 | Safety Data | Information regarding product safety, including warnings, |
| | | precautions, and safety protocols |
| 25 | Product Image | Visual representation of the product (e.g., photos or diagrams) |
| 26 | Preferred Supplier | The preferred or contracted supplier for the product, based on |
| | | cost or quality criteria |
| 27 | Lead Time | The time it takes from placing an order to receiving the |
| | | product from the supplier |
| 28 | Availability Status | Information about product availability, such as “In Stock”, |
| | | “Backordered”, or “Out of Stock” |
| 29 | Product Variants | Variants or alternative versions of the product (e.g., size |
| | | options, color options, or different material types) |
| 30 | Repackaging | For products that may be repackaged or modified before use |
| | Information | (e.g., medications or surgical kits), this data helps track how |
| | | items are sold or used |
| 31 | Tax Category | The applicable tax classification for the product (e.g., taxable, |
| | | non-taxable, exempt from tax) |
| 32 | Sterility Status | Information about whether the product is sterile or requires |
| | | sterilization (important for surgical instruments) |
| 33 | Lot/Serialization | An identifier to signal whether a product is lot controlled or |
| | Classification | serialized, which is critical for traceability of product |
| | | management and use, especially in case of recalls |
| 34 | Product Recall | Historical data on any recalls or quality issues related to the |
| | Information | product, helping hospitals manage potential risks |
| 35 | Alternative/Similar | Products that serve as alternatives to the current product (e.g., |
| | Products | if the |
| | | item is out of stock or discontinued) |
| No. | Attribute Name | Description |
| 36 | Medical Device | Unique identifier for medical devices required by regulatory |
| | Identification | bodies like the FDA for tracking and reporting purposes |
| | (UDI) | |
| 37 | Treatment/Use | Standard usage instructions or guidelines for using the product |
| | Guidelines | in clinical or medical settings (e.g., “For external use only”) |
| 38 | Warranty | Warranty details, such as duration, terms, and coverage for the |
| | Information | product (e.g., “5-year warranty”) |
| 39 | Environmental | Sustainability or environmental impact attributes, especially |
| | Impact Data | relevant for products with eco-friendly certifications or |
| | | considerations |

The distributor PIM 174 may manage and track supplier-specific data that may relate to the distribution and supply of product items 140 across the healthcare supply chain. The distributor PIM 174 may manage data PIM data 10 that may allow the distributor PIM 174 to manage transactions such as purchase orders 152 and sales orders 154 including buying product items 140 from manufacturers and selling product item 140 to the providers. The distributor PIM 174 may include one or more of the attributes from Table 1A as required by the distributor, and may also include one or more of the following attributes from Table 1B:

TABLE 1B
Description of attributes of PIM data 150 for use with the distributor PIM 174.

| No. | Attribute Name | Description |
|---|---|---|
| 40 | Sub Brand Name | Refers to a name used to represent a specific product line, |
| | | service offering, or segment of a larger brand (often referred to |
| | | as the “parent brand”), and is a way to distinguish a particular |
| | | set of products or services under the umbrella of the main |
| | | brand while maintaining a connection to the parent brand's |
| | | reputation and identity |
| 41 | Global Location | The GLN is a 13-digit number used to uniquely identify |
| | Number (GLN) | physical locations, legal entities, or other important business |
| | | entities involved in the supply chain, and is primarily used for |
| | | identifying locations such as warehouses, distribution centers, |
| | | suppliers, retailers, or even business entities such as hospitals |
| | | or manufacturers |
| 42 | Global Product | The GPC Code is a numerical identifier that represents a |
| | Classification | specific product category in the GPC hierarchy. Similar to |
| | (GPC) | other classification systems like UNSPSC, the GPC Code is |
| | | structured in a hierarchical manner, starting with a broad |
| | | product classification and narrows down to more specific |
| | | product categories |
| 43 | Ordering Lead | The typical time required for the manufacturer to fulfill an |
| | Time | order and deliver the product to the distributor |
| No. | Attribute Name | Description |
| 44 | Supplier | Certifications such as ISO, FDA approvals, or others that |
| | Certification | validate the supplier's credibility and quality control processes |
| 45 | Supplier Data | The DUNS (Data Universal Numbering System) is a unique |
| | Universal | nine-digit identifier assigned to businesses and organizations |
| | Numbering | by Dun & Bradstreet (D&B), a global provider of business |
| | System (DUNS) | information. The DUNS number is widely used for identifying |
| | | and verifying business entities in various sectors, including |
| | | supply chain management, credit reporting, government |
| | | contracting, and corporate relationships |
| 46 | Quality Assurance | Supplier's quality assurance and control protocols, including |
| | Information | any certifications or standards they adhere to. |
| 47 | Sustainability and | Data on the supplier's sustainability practices, including their |
| | Environmental | environmental footprint, eco-friendly packaging, and |
| | Impact | adherence to environmental regulations |
| 48 | Supplier Diversity | Information regarding the supplier's commitment to diversity |
| | Information | in sourcing (e.g., minority-owned, women-owned, veteran- |
| | | owned business certifications) |
| 49 | Supplier Risk | Risk profile based on various factors like financial stability, |
| | Assessment | geopolitical location, and market reputation. This helps in |
| | | assessing the reliability of the supplier over time |

The supplier PIM 176 may include comprehensive data relating to product items 140 of the manufacturer that the manufacturer produces and sells. The supplier PIM 176 may, for example, include information about the organization ID 132 of the manufacturer, the product ID 130, certifications, compliance, and operational details that ensure quality, traceability, and regulatory adherence at an individual product level. The supplier PIM 176 may include one or more attributes from Table 1A and Table 1B as required by the supplier, and may also include one or more of the following attributes from Table 1C:

TABLE 1C
Description of PIM data 150 attributes for use with the supplier PIM 176.

| No. | Attribute Name | Description |
|---|---|---|
| 50 | Production | The manufacturer's production capacity, usually represented as |
| | Capacity | output per day, week, or month (important for procurement |
| | | planning and risk management) |
| 51 | Production Lead | The average time required by the manufacturer to produce and |
| | Time | deliver a product after an order is placed |
| 52 | Manufacturer's | Details on the manufacturer's strategy for handling supply |
| | Business | chain disruptions, including natural disasters, pandemics, or |
| | Continuity Plan | other emergencies |
| 53 | Supplier Diversity | Data related to the manufacturer's involvement in diversity |
| | | initiatives, such as minority-owned, women-owned, or |
| | | veteran-owned business certifications |
| 54 | Manufacturing | Manufacturing Location(s): The specific site(s) where the |
| | Process and | product or its components are manufactured, critical for |
| | Compliance | ensuring compliance with local regulations and industry |
| | Information | standards |
| 55 | Quality Assurance | Quality Control Processes: Details of the manufacturer's |
| | and Control | internal quality control processes, including inspections, |
| | Information | testing standards, and methods for ensuring product quality. |
| | | Product Testing Protocols: Information on testing and quality |
| | | checks performed on the product during and after production |
| | | (e.g., “Sterility Testing”, “Tensile Strength Test”) |
| 56 | Manufacturer | Contact details for the manufacturer, including email, phone |
| | Contact | number, and address |
| | Information | |
| 57 | Manufacturer | Physical location(s) of the manufacturing facility, which could |
| | Address | include regional offices or production plants |

As shown in FIG. 9C, the PIM module 116 may match the product ID 130 and product item 140 to an existing product ID 130 and product item 140 in the system PIM 170. The PIM module 116 may include an enhanced provider PIM 178, an enhanced supplier PIM 180, and an enhanced distributor PIM 182, combined providing a comprehensive set of product item 140 information including the original product item 140 information from the organization, meta data 158 from third parties and PIM data 150, exhausting all potential PIM data 150 attributes that organizations could have within each local PIM system. More specifically, the PIM data 150 may enable the system 100 to align attributes that will be necessary to translate a product item 140 and insights from organization to organization across the system 100. The PIM module 116 may, for example, store in the database 108 multiple entries of the same product item 140 in order to account for a different supplier ID 166 for each instance of the product item 140. The enhanced provider PIM 178, enhanced supplier PIM 180, and enhanced distributor PIM 182 may enable organizations to implement changes to external PIM systems. The enhanced provider PIM 178, enhanced supplier PIM 180, and enhanced distributor PIM 182 may provide a central repository for a variety of product-related data, ensuring that the information is structured, consistent, and easily accessible.

As shown in FIG. 9C, the substitution module 118 may allow for allow for a system PIM 170 to include a substitute list 184 of product items 140 that may include the same or similar attributes as the product item 140. The substitution module 118 may also allow for the integration of a substitute list 184 provided by an organization, adding information relating to a substitute item 186 to an existing system PIM 170. The substitution module 118 may provide the substitute list 184 from the system PIM 170 or from an organization to match the product items 140 on the substitute list 184 to desired product item 140. The substitution module 118 may cross-reference both the system PIM 170 substitute list 184 and the substitute list 184 from the organization. The substitution module 118 may also allow organizations to provide feedback 187 confirming that the substitution is, in fact, a correct substitute product item 140. It should be appreciated that the cross-referencing of both the system PIM 170 substitute list 184 and the substitute list 184 from the organization, and substitute item 186 may create a higher level of confidence in identifying when product items 140 are the exact match or when product items 140 include similar attributes but are different product items 140.

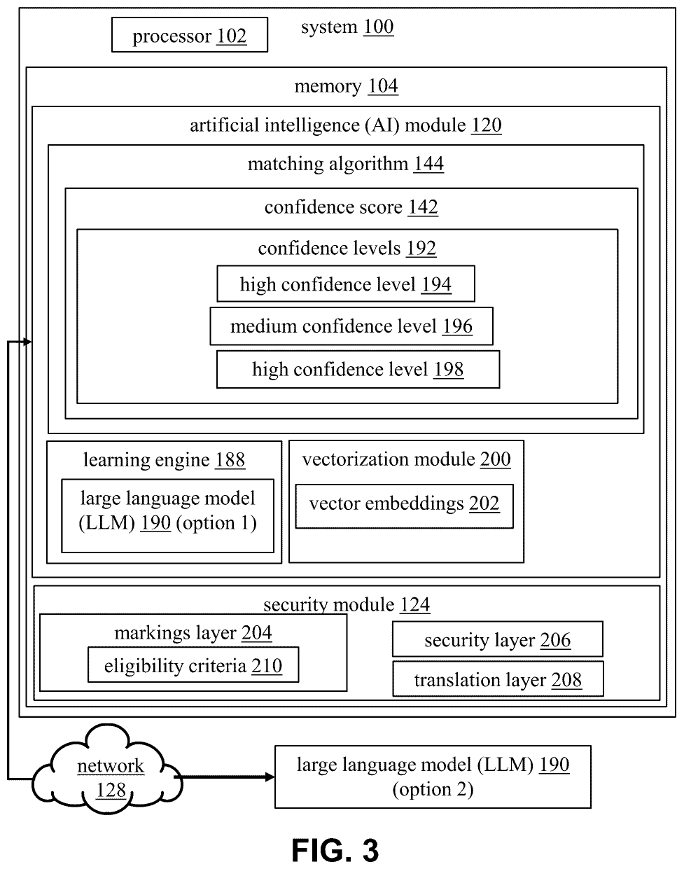

As shown in FIG. 3, the AI module 120 may enhance the product item 140 matching capabilities of system 100. The AI module 120 may receive the product ID 130 from the integration module 110. The AI module 120 may retrieve two or more existing product items 140 that include a partial match 138 to the product ID 130. The AI module 120 may integrate the product ID 130 with one of the two or more existing product items 140. The AI module 120 may include a learning engine 188 and a matching algorithm 144. The AI module 120 may include a large language model (LLM) 190. The LLM 190 may process the product ID 130 or an organization ID 132 to produce new product item 140 based on results of the partial match 138 of existing product items 140. Through the matching algorithm 144, the AI module 120 may facilitate the process for creating a new system PIM 170 or adding a product item 140 or attributes to an existing system PIM 170. The AI module 120 may, for example, use natural language processing (NPL) to fine-tune the LLM 190, vectorize PIM data 150 relating to a product ID 130 or an organization ID 132, or generate and store vector embeddings 202 in the database 108. The AI module 120 may include a local LLM 190, as shown in FIG. 3, option 1, or may utilize a remote LLM 190 via a network 128 as shown in FIG. 3, option 2. It should be understood that the AI module 120 may be periodically trained and fine-tuned with new PIM data 150 from the organization to identify a wide range of data to accurately generate the vector embeddings 202. The learning engine 188 may match the product ID 130 with one of the two or more existing product items 140 based on the partial match 138 to the product ID 130. The learning engine 188 may also receive a confidence score 142 from the matching algorithm 144 to match the product ID 130 to one of the two or more existing product items 140 based on the confidence score 142.

The matching algorithm 144 may generate a confidence score 142 based on the partial match 138 between the product ID 130 and the two or more existing product items 140. The matching algorithm 144 may provide the confidence score 142 to the learning engine 188 for matching the product ID 130 based on the confidence score 142. The matching algorithm 144 may evaluate two or more existing product items 140 based on several factors, including, for example, product item 140 attributes compiled from organizations, vectorization and meta data 158 enrichment. Factors to consider may also include transactional information such as purchase orders 152 and sales orders 154, and information based on substitute lists 184, allowing for further examination of matching products across organizations who transact with each other. Each product item 140 may be assigned a confidence score 142 based on how the attributes of the product item 140 align with existing product items 140 in the system 100.

The confidence score 142 may follow a tiered approach to determine the level of matching qualities between product items 140, guiding the decision-making process for whether a product item 140 matches an existing product item 140 or should be added to the system 100 as a new system PIM 170. For example, the matching algorithm 144 may analyze PIM data 150 attributes of the product item 140 such as attribute no. 15—GTIN, attribute no. 3—the description of the product item 140, attribute no. 4—manufacture names, and substitute lists 184. The confidence score 142 may include one or more confidence levels 192, including a high confidence level 194, a medium confidence level 196, and a low confidence level 198.

When the confidence score 142 includes a high confidence level 194, the AI module 120 may conclude that the product item 140 in question matches an existing product item 140 in the system 100. For example, the high confidence level 194 may be a result of comparing factors such as product item 140 attributes, purchase orders 152, sales orders 154, and substitute lists 184. The high confidence level 194 may be, for example, a confidence level 192 equal to or greater than 98%. The AI module 120 may confirm the matching product items 140 in the instance of a high confidence level 194, in which no further validation is needed, and the product item 140 may be considered aligned the PIM data 150 of the existing product item 140, ensuring efficiency and reducing manual intervention. However, if AI module 120 determines that a variation between the product item 140 PIM data 150 attributes and the PIM data 150 attributes of the existing product item 140, any attributes not matching will be stored in the database 108 for future use with the matching algorithm 144.

When the confidence score 142 includes a medium confidence level 196, the AI module 120 may conclude that a partial match 138 exists between the product item 140 and an existing product item 140 or PIM data 150, but the partial match 138 is not definitive. For example, the medium confidence level 196 may be a one or more confidence levels 192 ranging from 70% to 97% matching to an existing product item 140 or PIM data 150. For a medium confidence level 196, the matching algorithm 144 may involve human interaction to validate whether the product item 140 in question matches the product item 140 already in the system PIM 170. A manual review of the PIM data 150 attributes, such as attribute no. 3—description, and other key factors of the product item 140 may be required to confirm or deny that the partial match 138 does, in fact, belong in the system PIM 170. If the matching algorithm 144 confirms that the product items 140 match, a system 100 personnel or an organization may validate the incorporation of additional PIM data 150 attributes. If the product item 140 is not confirmed by the matching algorithm 144, the system 100 personnel or an organization may manually add the product item 140 to the PIM data 150.

When the confidence score 142 includes a low confidence level 198, the AI module 120 may conclude that no matching product items 140 exist in the system 100 compared with the product item 140 in question. In other words, there may be a discrepancy between the product item 140 in question and the PIM data 150 of an existing system PIM 170. For example, the low confidence level 198 may be a confidence level 192 less than or equal to 69% matching with an existing product item 140 or PIM data 150. The confidence score 142 may then prompt the system 100 to generate a new system PIM 170 based on the product item 140 in question. It should be appreciated that the tiered approach of the matching algorithm 144 may provide organizations with an assurance that communications with other organizations are about the same product item 140 even though that product item 140 may have unique descriptions and attributes in each PIM system for each organization.

As shown in FIGS. 3 and 9A-9B, the AI module 120 may include a vectorization module 200 to vectorize PIM data 150. For PIM data 150 to be consistently and efficiently shared, interpreted, and integrated across different organizations, the AI module 120 may normalized, vectorized, and store the PIM data 150 in the database 108 as a vector for use in the LLM 190. In other words, the vectorization module 200 may convert the raw text data of the product item 140, such as such as product descriptions or specifications, into numerical representations for the LLM 190 to easily retrieve and analyze. The vector embeddings 202 may capture semantic relationships between words, phrases, or product attributes of the PIM data 150 of a product item 140, allowing the AI module 120 to easily compare, match, and interpret PIM data 150 across organizations. It should be appreciated that the vector embeddings 202 may militate against inconsistent terminology, for example, when organizations use different naming conventions for the same product item 140 or PIM data 150 attributes. For example, a certain supplier may refer to a product item 140 as “Sterile Surgical Gloves,” while another supplier may use the terms “Surgical Gloves”, “Gloves, Surgical”, or “Single Sterile Gloves.” Without the use of vector embeddings 202, reconciling PIM data 150 from different sources may be complex and error-prone. The vector embeddings 202 may embedded directly into the normalized PIM data 150 within the database 108, allowing each product item 140 to retain its semantic meaning within the system 100. When new PIM data 150 is added or updated, e.g., a new product item 140 description, specification, or third-party data, the vectorization module 200 may automatically re-vectorize and enrich the PIM data 150, ensuring the latest version of the product item 140 may be accurately represented.

As shown in FIGS. 1 and 3, the validation module 122 may receive the confirming ID 134 from the communication module 112. The validation module 122 may validate the product ID 130 based on the confirming ID 134. The validation module 122 may also validate the product ID 130 based on a transactional data 146 generated between two or more organization accounts 148, including purchase orders 152 and sales orders 154. For example, the validation module 122 may validate a product ID 130 or a product item 140 by utilizing a GTIN provided by an organization or retrieved from an authorized database 136 such as Global Standards (GS1). For example, two or more organizations may utilize the same GTIN, among other unique attributes, to distinguish a product item 140, allowing the validation module 122 to validate the GTIN for integration with the system PIM 170. The validation module 122 may also retrieve information from an authorized database 136, e.g., a medical device database, accessed through the FDA such as Global Unique Device Identification Database (GUDID), an authorized database 136 containing key device identification information submitted to the FDA about medical devices that have Unique Device Identifiers (UDI). The validation module 122 may utilize enterprise resource planning (ERPs), or customer related information. The validation module 122 may rely upon transactional data 146 stored in the database 108, for example, purchase orders 152, sales orders 154, shipment and/or advanced shipping notices in order to validate a product ID 130 or a product item 140. The validation module 122 may also utilize substitute items 186 to validate a product ID 130. It should be appreciated that the validation module 122 may offer transparency in product matching workflows, enabling a “human in the loop” approach to ensure alignment to product item 140 matching decisions made by the AI module 120.

As shown in FIGS. 9A-9C, the security module 124 may protect against unauthorized access and manipulation of PIM data 150. The security module 124 may include a markings layer 204, a security layer 206, and a translation layer 208. The Markings layer 204 may provide an initial level of access control to product items 140 and PIM data 150 attributes, as shown in FIG. 9A. The markings layer 204 may determine eligibility criteria 210 for an organization, granting or restricting visibility and permission to change or add to PIM data 150 based on the eligibility criteria 210. In other words, to access a product item 140, PIM data 150 attributes, or data associated with an organization ID 132, an organization must pass all eligibility criteria 210 in order to bypass the markings layer 204 and obtain access. The markings layer 204 may be intended to manage when organizations may access any given authorized category of data. While the markings layer 204 may provide mandatory initial access control, internal organizational roles of who may access what data or objects may be discretionary. It should be understood that the markings layer 204 may allow the data from different organizations to be secure in separate datasets.

As shown in FIG. 9B, the security layer 206 may provide an additional protection for the product item 140 and PIM data 150, decoupling access for each organization so that organizations may not view or manipulate data relating to the matching algorithm 144. In other words, the system 100 may maintain the matching algorithm 144 in a separate ecosystem that may be protected from being changed, enhancing the authenticity of the decision making of the AI module 120 relating to the matching algorithm 144. The security layer 206 may allow organizations to access only the data relating to each respective organization or data that the organization may have been granted access to from other organizations.

As shown in FIG. 9C, the translation layer 208 may provide is a notional barrier within the platform to separate data objects that represent specific product information and data objects that enable a higher level of confidence in creating the common language of the product information across the organizations in the platform. The platform not only maintains product information from each organization, but also other supply chain information that can be used to validate product information even further.

As shown in FIGS. 11A and 11B, a method 300 for managing healthcare supply chain product and organization information for an organization is provided. The method 300 may include a step 302 of providing a processor 102 and a memory 104 in communication with the processor 102. The memory 104 may include a user interface module 106 that receives a product ID 130 and an organization ID 132 from the organization. The memory may include an integration module 110 that receives the product ID 130 and organization ID 132 from the user interface module 106. The integration module 110 may generate a new product item 140 when the integration module 110 determines that the product ID 130 is not associated with an existing product item 140. The integration module 110 may generate a new organization account 148 when the integration module 110 determines that the organization ID 132 is not associated with an existing organization account 148. The integration module 110 may integrate the product ID 130 with the existing product item 140 when the existing product item 140 does not include the product ID 130. The integration module 110 may integrate the organization ID 132 with the existing organization account 148 when the existing organization account 148 does not include the organization ID 132. The memory may include an organization module 114 that receives the organization ID 132 associated with the organization ID 132 from the integration module 110. The organization module 114 may match the organization ID 132 and organization account 148 to an organization master 160. The memory may include an PIM module 116 that receives the product item 140 associated with the product ID 130 from the integration module 110. The PIM module 116 may match the product ID 130 and product item 140 to a system PIM 170.

The method 300 may include a step 304 of receiving the product ID 130 and the organization ID 132 from the organization. The method 300 may include a step 306 of generating the new product item 140 when the integration module 110 determines that the product ID 130 is not associated with an existing product item 140. The method 300 may include a step 308 of generating the new organization account 148 when the integration module 110 determines that the organization ID 132 is not associated with an existing organization account 148. The method 300 may include a step 310 of integrating the product ID 130 with the existing product item 140 when the existing product item 140 does not include the product ID 130. The method 300 may include a step 312 of integrating the organization ID 132 with the existing organization account 148 when the existing organization account 148 does not include the organization ID 132. The method 300 may include a step 314 of matching the organization ID 132 and organization account 148 to an organization master 160. The method 300 may include a step 316 of matching the product ID 130 and product item 140 to a system PIM 170.

As shown in FIG. 12, a method 400 for managing healthcare supply chain product and organization information for an organization is provided. The method 400 may include steps 302-312 of method 300 (as steps 402-412 respectively). The method 400 may include a step 414 of providing in the memory 104 an organization module 114 that may generate a new organization account 148 or integrate an alias 168 into the existing organization account 148 that includes a member selected from a group consisting of a provider identifier, a distributor ID 164, or a supplier ID 166. The method 400 may include a step 416 of generating a new organization account 148 that includes a member selected from the group consisting of a provider identifier, a distributor ID 164, or a supplier ID 166. The method 400 may include a step 418 of integrating an alias 168 into the existing organization account 148. The method 400 may include steps 314-316 of method 300 (as steps 420-422 respectively).

As shown in FIG. 13, a method 500 for managing healthcare supply chain product and organization information for an organization is provided. The method 500 may include steps 302-308 of method 300 (as steps 502-508 respectively). The method 500 may include a step 510 of providing in the memory a database 108 to store the product ID 130, the organization ID 132, the new product item 140, and the new organization account 148. The method 500 may include a step 512 of storing the product ID 130 and the organization ID 132 via the database 108. The method 500 may include a step 514 of storing the new product item 140 and the new organization account 148 via the database 108. The method 500 may include steps 310-316 of method 300 (as steps 516-522 respectively).

As shown in FIG. 14, a method 600 for managing healthcare supply chain product and organization information for an organization is provided. The method 600 may include steps 302-304 of method 300 (as steps 602-604 respectively). The method 600 may include a step 606 of providing in the memory a communication module 112 to retrieve a confirming ID 134 from an authorized database 136 for generating a new product item 140 based on the product ID 130 and the confirming ID 134, and a validation module 122 to receive the confirming ID 134 from the communication module 112 and validate the product ID 130 based on the confirming ID 134 or on a transactional data 146 generated between two or more organization accounts 148 148. The method 600 may include a step 608 of retrieving a confirming ID 134 from an authorized database 136 via the communication module 112. The method 600 may include a step 610 of receiving from the communication module 112 the confirming ID 134 via the validation module 122. The method 600 may include a step 612 of validating the product ID 130 via the validation module 122 based on the confirming ID 134. The method 600 may include a step 614 of validating the product ID 130 based on a transactional data 146 generated between two or more organization accounts 148 148. The method 600 may include steps 306-316 of method 300 (as steps 616-626 respectively).

As shown in FIG. 15, a method 700 for managing healthcare supply chain product and organization information for an organization is provided. The method 700 may include steps 302-308 of method 300 (as steps 702-708 respectively). The method 700 may include a step 710 of providing in the memory an artificial intelligence (AI) module to receive the product ID 130 from the integration module 110, retrieve two or more existing product items 140 that include a partial match 138 to the product ID 130, and integrate the product ID 130 with one of the two or more existing product items 140. The method 700 may include a step 712 of receiving the product ID 130 from the integration module 110. The method 700 may include a step 714 of retrieving two or more existing product items 140 via the AI module 120 that include the partial match 138 to the product ID 130. The method 700 may include a step 716 of integrating the product ID 130 with one of the two or more existing product items 140 via the AI module 120. The method 700 may include steps 310-316 of method 300 (as steps 718-724 respectively).

As shown in FIG. 16, a method 800 for managing healthcare supply chain product and organization information for an organization is provided. The method 800 may include steps 302-308 of method 300 (as steps 802-808 respectively). The method 800 may include a step 810 of including in the AI module 120 a learning engine 188 to match the product ID 130 with one of the two or more existing product items 140 based on the partial match 138 to the product ID 130, and a matching algorithm 144 to generate a confidence score 142 based on the partial match 138 between the product ID 130 and the two or more existing product items 140, where the learning engine 188 may match the product ID 130 to one of the two or more existing product items 140 based on the confidence score 142. The method 800 may include a step 812 of matching the product ID 130 with one of the two or more existing product items 140 based on the partial match 138 to the product ID 130 via the matching algorithm 144. The method 800 may include a step 814 of generating a confidence score 142 via the matching algorithm 144 based on the partial match 138 between the product ID 130 and the two or more existing product items 140 and determining whether the confidence score 142 results in a high confidence level 194. The method 800 may include a step 816 of matching the product ID 130 to one of the two or more existing product items 140 via the learning engine 188 when the confidence score 142 results in a high confidence level 194. The method 800 may include steps 310-316 of method 300 (as steps 818-824 respectively).

As shown in FIGS. 9A-9D and 17A-17B, a method 900 for managing healthcare supply chain product and organization information for an organization is provided. The method 900 may include step 902 of providing a processor 102 and a memory 104 in communication with the processor 102. The memory 104 may include a user interface module 106, a database 108, an integration module 110, a communication module 112, an organization module 114, a PIM module 116, a substitution module 118, an artificial intelligence (AI) module 120, a validation module 122, and a security module 124. The user interface module 106 may receive a product ID 130 and an organization ID 132 from an organization. The integration module 110 may generate a new product item 140 when the integration module 110 determines that the product ID 130 is not associated with an existing product item 140. The integration module 110 may generate a new organization account 148 when the integration module 110 determines that the organization ID 132 is not associated with an existing organization account 148. The integration module 110 may integrate the product ID 130 with the existing product item 140 when the existing product item 140 does not include the product ID 130. The integration module 110 may integrate the organization ID 132 with the existing organization account 148 when the existing organization account 148 does not include the organization ID 132. The integration module 110 may normalize all data provided by the organization. The communication module 112 may receive information from the external database 156 to enrich PIM data 150 by providing additional information, including third-party meta data 158, embedding the meta data 158 directly into the PIM data 150. The organization module 114 may receive the organization ID 132 associated with the organization ID 132 from the integration module 110 and may match the organization ID 132 and organization account 148 to an organization master 160. The PIM module 116 may receive the product item 140 associated with the product ID 130 from the integration module 110. The PIM module 116 may match the product ID 130 and product item 140 to an system PIM 170. The substitution module 118 may allow for allow for a system PIM 170 to include a substitute list 184 of product items 140 that may include the same or similar attributes as the product item 140. The AI module 120 may include a learning engine 188 and a matching algorithm 144, utilizing a confidence score 142. The AI module 120 may include a vectorization module 200 to generate vector embeddings 202 from PIM data 150 provided by the organization. The validation module 122 may validate the product ID 130 based on the confirming ID 134, or on transactional data 146. The security module 124 may protect against unauthorized access and manipulation of PIM data 150 and may include a markings layer 204, a security layer 206, and a translation layer 208.

The method 900 may include a step 904 of receiving PIM data 150 from the organization and store the PIM data 150 in the database 108. The AI module 120 may automatically analyze the PIM data 150 to understand the local PIM system of the organization. The method 900 may include a step 906 of normalizing the PIM data 150 to allow for consistent data management through the system 100. The method 900 may include a step 908 of vectorizing the PIM data 150 provided by the organization, creating a vector embeddings 202. The method 900 may include a step 910 of retrieving meta data 158 from an external database 156 to add attributes to the PIM data 150. The method 900 may include a step 912 of analyzing transactional data 146 including purchase orders 152 and sales orders 154. The method 900 may include a step 914 of creating a confidence score 142 via the matching algorithm 144 using the vector embeddings 202, the meta data 158, and transactional data 146 to determine if the PIM data 150 matches an existing system PIM 170. The method 900 may include a step 916 of integrating the PIM data 150 into the system PIM 170 if the AI module 120 determines that the PIM data 150 and the system PIM 170 match and creating a new system PIM 170 if the AI module 120 determines that PIM data 150 and the system PIM 170 do not match. The method 900 may include a step 918 of supplementing the local PIM system of the organization with the PIM data 150 or new system PIM 170.

The system 100 may include a non-transitory computer-readable medium 212, operable to store processor instructions 214 for managing a healthcare supply chain for an organization. When executed by a processor 102, the processor instructions 214 may cause the processor 102 to receive a product ID 130 and an organization ID 132 from the organization. The processor instructions 214 may cause the processor 102 to generate a new product item 140 when an integration module 110 determines that the product ID 130 is not associated with an existing product item 140. The processor instructions 214 may cause the processor 102 to generate a new organization account 148 when the integration module 110 determines that the organization ID 132 is not associated with an existing organization account 148. The processor instructions 214 may cause the processor 102 to integrate the product ID 130 with the existing product item 140 when the existing product item 140 does not include the product ID 130. The processor instructions 214 may cause the processor 102 to integrate the organization ID 132 with the existing organization account 148 when the existing organization account 148 does not include the organization ID 132. The processor instructions 214 may cause the processor 102 to match the organization ID 132 and organization account 148 to an organization master 160. The processor instructions 214 may cause the processor 102 to match the product ID 130 and product item 140 to a system PIM 170.

Advantageously, the disclosed healthcare supply chain management method and system 100 addresses the significant challenges identified in the prior art, such as inefficiencies, lack of real-time data visibility, and the inability to effectively manage disruptions within the healthcare supply chain. By integrating advanced technologies like machine learning and regular communication with third party external databases 156, the system 100 enhances data collection, integration, analysis, and interaction processes across various stakeholders including manufacturers, suppliers, distributors, and providers. The integration of the system 100 with the PIM system of an organization may allow for a seamless flow of information and facilitate the generation of actionable insights that improve decision-making. Furthermore, the capability of the system 100 to enrich organization PIM systems with comprehensive healthcare utilization data, enabling more accurate forecasting and demand planning. Overall, the present technology may significantly militate against the issues of manual data review, slow response times, and the fragmented nature of other supply chain systems, thereby optimizing healthcare supply chain operations and enhancing patient care outcomes.

### EXAMPLES

Example embodiments of the present technology are provided with reference to the FIGS. 1-17B enclosed herewith.

### Example 1: Multi-Enterprise Product Translation Between Healthcare Provider and Supplier

A healthcare provider organization may utilize the system 100 to translate product item 140 information with a medical device supplier while maintaining their unique internal product terminology. The local PIM system of the provider may contain PIM data 150 with specific product IDs 130 and descriptions, while the supplier's local PIM system may maintain different product attributes and naming conventions for the same product item 140. The AI module 120 may execute the normalization process on the PIM data 150 to create standardized data structures from both organizations' disparate product information. The AI module 120 may generate vector embeddings 202 to capture semantic relationships between the provider's and supplier's product descriptions, enabling accurate product item 140 matching despite different terminologies.

The communication module 112 may facilitate communication with an external database 156 such as FDA registrations and DUNS numbers to enrich the product data with authoritative third-party meta data 158. The system 100 may store the PIM data 150 that combines the original organizational product item 140 information with the enriched meta data 158 from regulatory sources. The organization module 114 may maintain separate provider IDs 162 and supplier IDs 166 while the PIM module 116 may create unified PIM data 150 in the system PIM 170. The validation module 122 may ensure data integrity throughout the translation process while the security module 124 may implement tiered security controls to protect each organization's private information.

The matching algorithm 144 within the AI module 120 may analyze transactional data 146 including purchase orders 152 and sales orders 154 to validate product relationships between the provider and supplier. The system 100 may generate a confidence score 142 based on multiple factors including PIM data 150 attributes, transactional data 146, and substitute lists 184 to determine match accuracy. When a confidence score 142 exceeds the high confidence level 194, the system 100 may automatically confirm product item 140 matches and update the system PIM 170 accordingly. The system 100 may facilitate real-time data synchronization between the provider's and supplier's systems while preserving each organization's unique product language and internal processes.

The substitution module 118 may leverage existing substitute lists 184 from both organizations and the system 100 to enhance product matching accuracy. The system 100 may identify when product IDs 130 may be exact matches versus similar but distinct product items 140 through comprehensive substitution analysis. The database 108 may maintain normalized PIM data 150 that enables seamless translation of PIM data 150 attributes between the provider's surgical mask specifications and the supplier's corresponding product catalog entries. The user interface module 106 may present translation results to authorized personnel while maintaining appropriate access controls through the security module 124. The system 100 may enable bidirectional data flow, allowing both the provider and supplier to enrich their local PIM systems with validated product item 140 information from the ecosystem. The system 100 may update so that improvements and corrections may be reflected in each organization's native technology platforms.

### Example 2: Automated Product Onboarding for Healthcare Distributor

A healthcare distributor organization may implement the system 100 to automate the onboarding of new product item 140 from multiple suppliers while maintaining consistency across their extensive product catalog. The local PIM system of the distributor may receive product data from various suppliers' local PIM system with inconsistent formatting, naming conventions, and attribute completeness. The AI module 120 may execute the normalization process to standardize incoming PIM data 150, while the AI module 120 may apply vector embedding 202 techniques to create semantic representations of product item 140 descriptions and specifications, enabling intelligent product categorization and duplicate detection.

The communication module 112 may automatically connect with external metadata sources including GS1 GDSN networks, and regulatory compliance systems to enrich incoming PIM data 150 information. The system 100 may store enhanced PIM data 150 that combines supplier-provided attributes with authoritative third-party validation data. The organization module 114 may maintain separate supplier IDs 166 and aliases 168 while the PIM module 116 may create unified product item 140 records that eliminate duplicate entries across multiple supplier catalogs. The user interface module 106 may facilitate real-time data feeds from suppliers while the security module 124 may implement appropriate access controls to protect competitive supplier information.

The matching algorithm 144 may analyze new product submissions against the existing system PIM 170 using multiple validation methods including GTIN matching, correlation with transactional data 14, and substitution list 184 analysis. The system 100 may generate a confidence score 142 that determines whether new product items 140 may be added to the catalog or matched with existing product item 140. When a confidence score 142 falls within the medium confidence level 196, the validation module 122 may trigger human-in-the-loop review processes to ensure accurate product item 140 classification. The database 108 may maintain comprehensive product hierarchies that enable efficient catalog management and support advanced analytics for inventory optimization.

The substitution module 118 may automatically identify potential product item 140 alternatives and substitute item 186 based on attribute similarity and historical usage patterns. The system 100 may create dynamic substitute lists 184 that may be updated as new product item 140 may be onboarded and relationships may be established through transactional data 146 analysis. The AI module 120 may execute automated workflows that may reduce new product setup time from days to hours while maintaining data quality standards. The user interface module 106 may provide dashboard views that enable distributor personnel to monitor onboarding progress and review exception cases requiring manual intervention.

The AI module 120 may enable automatic enrichment of existing product item 140 records as new supplier information becomes available through the ecosystem. The system 100 may update source PIM systems with the most current and complete product information. The user interface module 106 may provide automated notifications to relevant stakeholders when critical product information may be updated, ensuring that procurement, inventory, and sales teams may have access to the latest product data.

### Example 3: Supply Chain Risk Management Through Product Standardization

A multi-hospital health system may deploy the system 100 to standardize product item 140 information across the hospital facilities while maintaining visibility into supply chain risks and product availability. The local PIM systems at different hospitals within the system 100 may contain inconsistent product item 140 data, duplicate entries, and incomplete supplier information that may complicate procurement decisions and inventory management. The AI module 120 may execute comprehensive data normalization across the system 100 and all local PIM systems to create a unified view of the health system's product item 140 portfolio. The AI module 120 may apply advanced matching algorithms 144 that may identify duplicate products across facilities and consolidate product records while preserving facility-specific preferences and contracts.

The integration module 110 may establish connections with suppliers and distributors to create real-time visibility into product item 140 availability, descriptions, and additional product attributes. The system 100 may store enhanced PIM data 150 that may include supplier risk ratings, manufacturing locations, and business continuity information critical for supply chain resilience. The organization module 114 may maintain comprehensive supplier and distributor master data with aliases 168 and alternative product IDs 130 that may enable rapid supplier switching during emergencies. The communication module 112 may interface with external databases 156 and regulatory systems to provide early warning of product item 140 recalls, compliance issues, or supply chain disruptions.

The matching algorithm 144 may analyze transactional data 146 including purchase orders 152 and sales orders 154 across the health system to identify usage patterns and demand forecasting opportunities. The system 100 may generate a confidence score 142 for product item 140 matches that may enable automatic consolidation of similar items while flagging potential substitution opportunities for review. The substitution module 118 may maintain dynamic substitute lists 184 that may be updated based on clinical outcomes data and physician preferences across the health system.

The database 108 may maintain comprehensive product item 140 genealogy information that may enable rapid identification of affected product items 140 during recall events or quality issues. The system 100 may provide automated alerts when products from high-risk suppliers or manufacturing locations may be identified in inventory or pending orders. The processor may execute predictive analytics workflows that may identify potential supply shortages based on supplier capacity, lead time trends, and demand patterns across the health system. The user interface module 106 may provide executive dashboards that may display supply chain risk metrics, standardization progress, and cost savings opportunities resulting from product consolidation efforts.

The PIM module 116 may enable the health system to maintain standardized product item 140 information while preserving facility-level customizations and clinical preferences. The system 100 may update source PIM systems across all ERP and materials management systems to ensure consistent PIM data 150 data throughout the health system's technology infrastructure. The security module 124 may implement role-based access controls at the organization's discretion that may allow facility managers to view their specific product data while enabling system-wide visibility for procurement and supply chain personnel.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A system for managing a healthcare supply chain for an organization, comprising:
a processor;
a memory in communication with the processor, the memory including a user interface module, an integration module, an organization module, and a product information management (PIM) module;
wherein:
the user interface module is configured to:
receive a product identifier and an organization identifier from the organization;
the integration module is configured to:
receive the product identifier and organization identifier from the user interface module;
generate a product item when the integration module determines that the product identifier is not associated with an existing product item;
generate an organization account when the integration module determines that the organization identifier is not associated with an existing organization account,
integrate the product identifier with the existing product item when the existing product item does not include the product identifier, and
integrate the organization identifier with the existing organization account when the existing organization account does not include the organization identifier;
the organization module is configured to:
receive the organization identifier from the integration module, and
match the organization identifier and organization account to an organization master; and
the PIM module is configured to:
receive the product item associated with the product identifier from the integration module, and
match the product identifier and product item to an item master.

2. The system of claim 1, wherein the organization module is further configured to generate a new organization account that includes a member selected from a group consisting of a provider identifier, a distributor identifier, a supplier identifier, and combinations thereof.

3. The system of claim 2, wherein the organization module is further configured to integrate an alias into the existing organization account, the alias including a member selected from the group consisting of a provider identifier, a distributor identifier, a supplier identifier, and combinations thereof.

4. The system of claim 1, wherein the memory further includes a database configured to store the product identifier, the organization identifier, the product item, and the organization account.

5. The system of claim 1, wherein the memory further includes a communication module configured to retrieve a confirming identifier from an authorized database for generating a new product item based on the product identifier and the confirming identifier.

6. The system of claim 5, wherein the memory further includes a validation module configured to receive the confirming identifier from the communication module and validate the product identifier based on the confirming identifier.

7. The system of claim 6, wherein the validation module is further configured to validate the product identifier based on transactional data generated between two or more organization accounts.

8. The system of claim 1, wherein the memory further includes an artificial intelligence (AI) module configured to:
receive the product identifier from the integration module;
retrieve two or more existing product items that include a partial match to the product identifier; and
integrate the product identifier with one of the two or more existing product items.

9. The system of claim 8, wherein the AI module further includes a learning engine configured to match the product identifier with one of the two or more existing product items based on the partial match to the product identifier.

10. The system of claim 9, wherein the AI module further includes a matching algorithm configured to generate a confidence score based on the partial match between the product identifier and the two or more existing product items and determine if the confidence score results in a high confidence level, the learning engine further configured to match the product identifier to one of the two or more existing product items when the confidence score includes the high confidence level.

11. A method for managing a healthcare supply chain for an organization, comprising:
providing a processor, a memory in communication with the processor, the memory including a user interface module, an integration module, an organization module, and a product information management (PIM) module,
wherein:
the user interface module is configured to:
receive a product identifier and an organization identifier from the organization,
the integration module is configured to:
receive the product identifier and organization identifier from the user interface module,
generate a product item when the integration module determines that the product identifier is not associated with an existing product item,
generate an organization account when the integration module determines that the organization identifier is not associated with an existing organization account,
integrate the product identifier with the existing product item when the existing product item does not include the product identifier, and
integrate the organization identifier with the existing organization account when the existing organization account does not include the organization identifier;
the organization module is configured to:
receive the organization identifier associated with the organization identifier from the integration module, and
match the organization identifier and organization account to an organization master, and
the PIM module is configured to:
receive the product item associated with the product identifier from the integration module, and
match the product identifier and product item to an item master;
receiving the product identifier and the organization identifier from the organization;
generating the product item when the integration module determines that the product identifier is not associated with an existing product item;
generating the organization account when the integration module determines that the organization identifier is not associated with an existing organization account;
integrating the product identifier with the existing product item when the existing product item does not include the product identifier;
integrating the organization identifier with the existing organization account when the existing organization account does not include the organization identifier;
matching the organization identifier and organization account to an organization master; and
matching the product identifier and product item to an item master.

12. The method of claim 11, wherein:
the organization module is further configured to generate a new organization account that includes a member selected from a group consisting of a provider identifier, a distributor identifier, a supplier identifier, and combinations thereof; and
the method further comprises:
generating a new organization account that includes a member selected from the group consisting of a provider identifier, a distributor identifier, a supplier identifier, and combinations thereof.

13. The method of claim 12, wherein:
the organization module is further configured to integrate an alias into the existing organization account, the alias including a member selected from a group consisting of a provider identifier, a distributor identifier, a supplier identifier, and combinations thereof; and
the method further comprises:
integrating an alias into the existing organization account.

14. The method of claim 11, further comprising:
providing in the memory a database configured to store the product identifier, the organization identifier, the product item, and the organization account;
storing the product identifier and the organization identifier via the database; and
storing the product item and the organization account via the database.

15. The method of claim 11, further comprising:
providing in the memory a communication module configured to retrieve a confirming identifier from an authorized database for generating a new product item based on the product identifier and the confirming identifier; and
retrieving a confirming identifier from an authorized database via the communication module.

16. The method of claim 15, further comprising:
providing in the memory a validation module configured to receive the confirming identifier from the communication module and validate the product identifier based on the confirming identifier;
receiving from the communication module the confirming identifier via the validation module; and
validating the product identifier via the validation module based on the confirming identifier.

17. The method of claim 16, wherein:
the validation module is further configured to validate the product identifier based on a transactional data generated between two or more organization accounts; and
the method further comprises:
validating the product identifier based on transactional data generated between two or more organization accounts.

18. The method of claim 11, further comprising:
providing in the memory an artificial intelligence (AI) module configured to receive the product identifier from the integration module, retrieve two or more existing product items that include a partial match to the product identifier, and integrate the product identifier with one of the two or more existing product items;
receiving the product identifier from the integration module;
retrieving two or more existing product items via the AI module that include the partial match to the product identifier; and
integrating the product identifier with one of the two or more existing product items via the AI module.

19. The method of claim 18, wherein:
the AI module includes a matching algorithm and a learning engine, the learning engine configured to match the product identifier with one of the two or more existing product items based on the partial match to the product identifier, and the matching algorithm configured to generate a confidence score based on the partial match between the product identifier and the two or more existing product items, the learning engine further configured to match the product identifier to one of the two or more existing product items based on the confidence score;
the method further comprises:
matching the product identifier with one of the two or more existing product items based on the partial match to the product identifier via the matching algorithm;
generating a confidence score via the matching algorithm based on the partial match between the product identifier and the two or more existing product items;
determining whether the confidence score results in a high confidence level; and
matching the product identifier to one of the two or more existing product items via the learning engine when the confidence score results in the high confidence level.

20. A non-transitory computer-readable medium, operable to store processor instructions for managing a healthcare supply chain for an organization that, when the processor instructions are executed by a processor, causes the processor to:
receive a product identifier and an organization identifier from the organization;
generate a product item when an integration module determines that the product identifier is not associated with an existing product item;
generate an organization account when the integration module determines that the organization identifier is not associated with an existing organization account;
integrate the product identifier with the existing product item when the existing product item does not include the product identifier;
integrate the organization identifier with the existing organization account when the existing organization account does not include the organization identifier;
match the organization identifier and organization account to an organization master; and
match the product identifier and product item to an item master.
